(19) United States
(12) Patent Application Publication
(10) Pub. No.: US 2018/0310877 A1
(43) Pub. Date: Nov. 1, 2018

(54) APPARATUS, SYSTEM AND METHOD FOR PAIN MONITORING

(71) Applicant: MEDASENSE BIOMETRICS LTD., Ramat Gan (IL)

(72) Inventors: Galit ZUCKERMAN STARK, Tel Aviv (IL); Dagan HARRIS, Hadera (IL)

(21) Appl. No.: 15/740,295

(22) PCT Filed: Jul. 4, 2016
(86) PCT No.: PCT/IL2016/050714

§ 371 Date: Dec. 27, 2017

Related U.S. Application Data
(60) Provisional application No. 62/188,664, filed on Jul. 5, 2015.

(51) Int. Cl. A61B 5/00; A61B 5/0205; A61B 5/0402; A61B 5/0476; A61B 5/0488; A61B 5/0496; A61B 3/11

(52) U.S. Cl. CPC A61B 5/4821; A61B 5/02055; A61B 5/0402; A61B 5/0476; A61B 5/04884; A61B 5/021; A61B 3/112; A61B 5/7264; A61B 5/7278; A61B 5/4839; A61B 5/0496

(57) ABSTRACT

A nociception monitoring device including at least one sensor configured to sense at least three physiological parameters of a patient, and a computing unit configured to receive the at least three physiological parameters and to compute a nociception scale (NS) value, indicative of a nociception level of the patient, based on an analysis of the at least three physiological parameters and/or features derived therefrom.

### TECHNICAL FIELD

The present disclosure relates generally to the field of pain monitoring, specifically to monitoring of nociception during anesthesia and pain management.

### BACKGROUND

Pain is an unpleasant sensation, ranging from slight discomfort to intense suffering, which is perceived with wide variability by different individuals. The American Pain Society has named pain “the 5th vital sign” in order to promote better awareness, assessment, and treatment. Since pain is a subjective phenomenon, its assessment is based mainly on subjective one-dimensional scales of self-evaluation by the patient. These validated scales are widely used, but can be affected by extremes of pain, mood, age, and culture, among others, and by the caregiver's own biases and attitudes towards pain. Moreover, their use is limited in the young, uncooperative, or cognitively disabled patient as well as in anesthetized patients.

Analgesia is the last major aspect of anesthesia without a dedicated monitor. Nociception is typically monitored indirectly by measuring physiological, hemodynamic and other parameters that are assumed to change in response to noxious stimulation due to sympathetic activation. The anesthesiologist needs to integrate these physiological parameters with clinical signs as a basis for analgesic treatment during surgery. Being a basically intuitive and subjective interpretation of clinical and physiological data, this is a very limited and even problematic foundation for guiding patient treatment possibly leading to under- or over-medication of the patients. Different tools for monitoring pain have been developed in attempts to overcome this problem. These include tools based on heart rate variability, heart or pulse rate, pulse amplitude, pupilometry and even imaging techniques (Cowen et al. Anaesthesia 2015). However, each tool has encountered difficulty in providing an accurate, reliable and objective estimate of the patient's pain. Heart rate variability can be influenced by numerous physiological and psychological conditions such as age, sex, medication, depth of anesthesia, emotions etc., and its accuracy in postoperative pain detection has been unconvincing. The surgical plethysmographic index has been shown to be able to distinguish strong noxious stimuli from no stimulation, but as being unable to consistently differentiate between stimulus intensities. The use of pupilometry in sedated patients is dubious, and imaging techniques are considered clinically impractical (Cowen et al. Anaesthesia 2015).

Accordingly, there remains a need for devices and methods enabling accurate nociception monitoring during surgery and/or anesthesia.

### SUMMARY

Aspects of the disclosure, in some embodiments thereof, relate to nociception monitoring during surgery and/or anesthesia.

Accurate measurement of nociception/analgesia during anesthesia remains a challenging task. During surgery, patients are anesthetized to avoid sensation of and response to noxious stimuli. However, due to the unconsciousness of the patient, the treating clinician must estimate the patient's level of nociception as a basis for analgesic treatment. Most anesthesia health care providers, if not all, use changes in heart rate and blood pressure as markers of the occurrence of acute nociceptive events. While these variables may suffice when intense nociceptive stimuli occur, mild and moderate stimuli are often not detected or detected too late.

Advantageously, the device and method, disclosed herein, enable identification of severe, moderate and even mild nociception levels. It is understood by one of ordinary skill in the art that failure to optimally manage analgesia (whether overdosing or lack thereof) may prolong and/or interfere with the patient's recovery from surgery. It is further understood that identification of mild nociception, for example during surgery, may enable its treatment at an earlier stage, thereby avoiding deterioration into severe pain that leads to physiological stress responses, prolonged recovery and even chronic pain.

Evidence has accumulated on the importance of sufficient analgesic during surgery, as surgery-induced inflammatory reaction and immune suppression may cause postoperative imbalance (e.g. cardiovascular, neurologic, renal, hepatic imbalance) and cancer growth. However, overdoses of analgesics are likewise associated with adverse event, poor outcome and patient discomfort. This emphasizes the need for continuous and accurate monitoring of nociception/analgesia in order to, on the one hand, prevent pain, delirium, infection, nausea and vomiting, and, on the other hand promote wound healing, fast rehabilitation, and discharge.

Advantageously, the method and device, disclosed herein, enable reliable differentiation between different levels of pain, which is a prerequisite for balanced pain management. Based on the accurate assessment of the patient's nociception level, need for an adjustment of analgesic type and/or dose may be determined.

Detection of nociception/analgesia during anesthesia is further challenging due to changes in various physiological parameters caused by drugs administered to the patient. For example, various anesthetics and analgesics have been shown to have hemodynamic effects, such as bradycardia and vasodilation, which in turn may interfere with the measurement of the physiological parameters related to pain, such as changes in heart rate, blood pressure and the like. As a result, nociception may easily be overlooked due to the physiological changes caused by drugs administered.

Advantageously, the device and method disclosed herein enable an objective assessment of the patient's nociception level during anesthesia and/or while being administered with analgesics in that the assessment of the nociception level is essentially unaffected by the physiological changes caused by the administered drugs. Thus, reliable nociception/analgesia monitoring during surgery, as well as other medical interventions, is provided.

As a result of the accurate and objective assessment of the patient's nociception level, the need for intensive medical attendance after surgery may be reduced. This may enable earlier release from post-operative and intensive care units to general floor and/or to home care facilities. Advantageously, the monitoring device, disclosed herein, enables monitoring nociception/analgesia using a small wearable monitor in the form of a finger probe, a wristwatch, wristbands, gloves or the like, making home-care post-operative recovery monitoring a real possibility.

According to some embodiments, there is provided a nociception monitoring device including at least one sensor adapted to sense at least three physiological parameters of a patient, and a computing unit. The computing unit is adapted to receive said at least three physiological parameters from said at least one sensor and to compute a nociception scale (NS) value based on an analysis of the at least three physiological parameters and/or features derived therefrom. According to some embodiments, the computing unit may be adapted to receive the at least three physiological parameters from the at least one sensor and to compute a nociception scale (NS) value based on an integrative analysis of the at least three physiological parameters and/or features derived therefrom. According to some embodiments, the NS value may be indicative of a nociception level of the patient; and may be essentially unaffected by vasodilating and/or bradycardial effects caused by administration of an analgesic and/or an anesthetic to the patient. According to some embodiments, is an NS value changed by less than 10 units

According to some embodiments, when the NS value crosses a first predefined threshold value a mild nociception level is identified, and when the NS value crosses a second predefined threshold value a severe nociception level is identified.

According to some embodiments, the patient is anesthetized. According to some embodiments, the patient is administered with an analgesic.

According to some embodiments, the NS value enables differentiation between no nociception, mild nociception and/or severe nociception in the patient administered with the analgesic.

According to some embodiments, the NS value enables differentiation between no nociception, mild nociception, moderate nociception and/or severe nociception in the patient administered with the analgesic.

According to some embodiments, the NS value provides regression of the nociception level into a numerical scale.

According to some embodiments, the at least one sensor is selected from a bio-potential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a us blood pressure sensor, a three-axis accelerometer, a diffused correlation spectroscopy (DCS) sensor, an acoustics sensor, a bio-impedance sensor and a piezoelectric sensor, an audio sensor, motion sensing input device or any combination thereof.

According to some embodiments, the at least one sensor is selected from a galvanic skin response (GSR) sensor, a plethysmography (PPG) sensor, a skin temperature sensor, and a three-axis accelerometer or any combination thereof.

According to some embodiments, the at least one sensor may include at least a galvanic skin response (GSR) sensor and a plethysmography (PPG) sensor.

According to some embodiments, the device may include at least three sensors. According to some embodiments, the at least three sensors may include at least a galvanic skin response (GSR) sensor, a plethysmography (PPG) sensor and a skin temperature sensor. According to some embodiments, the at least three sensors may include at least a galvanic skin response (GSR) sensor, a plethysmography (PPG) sensor and a three-axis accelerometer. According to some embodiments, the at least three sensors may include at least a plethysmography (PPG) sensor, a skin temperature sensor and a three-axis accelerometer. According to some embodiments, the at least three sensors may include at least galvanic skin response (GSR) sensor, a skin temperature sensor and a three-axis accelerometer. According to some embodiments, the at least three sensors may include at least a galvanic skin response (GSR) sensor, a plethysmography (PPG) sensor and an electrocardiograph (ECG). According to some embodiments, the at least three sensors may include at least a galvanic skin response (GSR) sensor, a plethysmography (PPG) sensor and an electroencephalograph (EEG). According to some embodiments, the at least three sensors may include at least a galvanic skin response (GSR) sensor, a plethysmography (PPG) sensor and an electrooculograph (EOG). According to some embodiments, the at least three sensors may include at least an electrocardiograph (ECG), a skin temperature sensor and a three-axis accelerometer. According to some embodiments, the at least three sensors may include at least a galvanic skin response (GSR) sensor, a plethysmography (PPG) sensor and a facial electromyograph (FEMG). According to some embodiments, the at least three sensors may include at least an electrocardiograph (ECG), a plethysmography (PPG) sensor and an electroencephalograph (EEG). According to some embodiments, the at least three sensors may include at least a galvanic skin response (GSR) sensor, a plethysmography (PPG) sensor and respiration sensor. According to some embodiments, the at least three sensors may include a galvanic skin response (GSR) sensor, a plethysmography (PPG) sensor, a skin temperature sensor, a three-axis accelerometer and an electroencephalograph (EEG). It is understood that other combinations of sensor may also be envisaged and is within the scope of the disclosure.

According to some embodiments, the at least three parameters are selected from heart/pulse rate (HR or PR), heart rate variability (HRV) monitor, amplitude of photoplethysmogram, skin conductance level (SCL), number of skin conductance fluctuations (NSCF), blood pressure, movement and any combination thereof.

According to some embodiments, the at least three physiological parameters may at least include heart rate/pulse rate (HR/PR), heart rate variability (HRV) and amplitude of photoplethysmogram. According to some embodiments, the at least three physiological parameters may at least include heart rate variability (HRV), amplitude of photoplethysmogram and skin conductance level (SCL). According to some embodiments, the at least three physiological parameters may at least include amplitude of photoplethysmogram, skin conductance level (SCL) and arterial blood pressure. According to some embodiments, the at least three physiological parameters may at least include heart rate variability (HRV), amplitude of photoplethysmogram, and movement. According to some embodiments, the at least three physiological parameters may at least include heart rate variability (HRV), amplitude of photoplethysmogram, skin conductance level (SCL), number of skin conductance fluctuations (NSCF). It is understood that other parameters and combination of parameters, such as any combination of the parameters and features disclosed in table 1 herein, may also be envisaged and are within the scope of the present disclosure.

According to some embodiments, the NS value may enable differentiation between no nociception, and nociception with a sensitivity of above 70% at a specificity of at least 70%. According to some embodiments, the NS value may enable differentiation between no nociception, and nociception with a sensitivity of above 70% at a specificity of at least 75%. According to some embodiments, the NS value may enable differentiation between no nociception, and nociception with a sensitivity of above 75% at a specificity of at least 75%. According to some embodiments, NS value may enable differentiation between no nociception and nociception with a sensitivity of above 80% at a specificity of at least 75%. According to some embodiments, the NS value may enable differentiation between no nociception, and nociception with a sensitivity of above 85% at a specificity of at least 75%. According to some embodiments, the NS value may enable differentiation between no nociception and nociception with a sensitivity of above 70% at a specificity of at least 84%. According to some embodiments, the NS value may enable differentiation between no nociception and nociception with a sensitivity of above 80% at a specificity of at least 84%. According to some embodiments, the NS value may enable differentiation between no nociception and nociception with a sensitivity of above 85% at a specificity of at least 84%. According to some embodiments, the NS value may enable differentiation between no nociception and mild nociception with a sensitivity of above 75% at a specificity of at least 75%.

According to some embodiments, there is provided a nociception monitoring device including at least one sensor adapted to sense at least three physiological parameters of a patient administered with an analgesics, and a computing unit adapted to receive the at least three physiological parameters from the at least one sensor, and to compute a nociception scale (NS) value based on an analysis of the at least three physiological parameters and/or features derived therefrom. According to some embodiments, the computing unit may be adapted to receive the at least three physiological parameters from the at least one sensor and to compute a nociception scale (NS) value based on an integrative analysis of the at least three physiological parameters and/or features derived therefrom. According to some embodiments, the NS value is capable of distinguishing between no nociception, mild nociception and/or severe nociception of the patient.

According to some embodiments, the NS value is essentially unaffected by vasodilating and/or bradycardial effects of analgesics and/or anesthetics.

According to some embodiments, the at least one sensor is selected from a bio-potential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a us blood pressure sensor, a three-axis accelerometer, a diffused correlation spectroscopy (DCS) sensor, an acoustics sensor, a bio-impedance sensor and a piezoelectric sensor, an audio sensor, motion sensing input device or any combination thereof.

According to some embodiments, the nociception monitoring device includes at least three sensors. According to some embodiments, the at least three sensors may include a PPG sensor, a GSR sensor and a three-axis accelerometer and/or a skin temperature sensor. According to some embodiments, the at least three sensors may include at least a plethysmography (PPG) sensor, a skin temperature sensor and a three-axis accelerometer. According to some embodiments, the at least three sensors may include at least galvanic skin response (GSR) sensor, a skin temperature sensor and a three-axis accelerometer. According to some embodiments, the at least three sensors may include at least a galvanic skin response (GSR) sensor, a plethysmography (PPG) sensor and an electrocardiograph (ECG). According to some embodiments, the at least three sensors may include at least a galvanic skin response (GSR) sensor, a plethysmography (PPG) sensor and an electroencephalograph (EEG). According to some embodiments, the at least three sensors may include at least a galvanic skin response (GSR) sensor, a plethysmography (PPG) sensor and an electrooculograph (EOG). According to some embodiments, the at least three sensors may include at least an electrocardiograph (ECG), a skin temperature sensor and a three-axis accelerometer. According to some embodiments, the at least three sensors may include at least a galvanic skin response (GSR) sensor, a plethysmography (PPG) sensor and a facial electromyograph (FEMG). According to some embodiments, the at least three sensors may include at least an electrocardiograph (ECG), a plethysmography (PPG) sensor and an electroencephalograph (EEG). According to some embodiments, the at least three sensors may include at least a galvanic skin response (GSR) sensor, a plethysmography (PPG) sensor and respiration sensor. According to some embodiments, the at least three sensors may include a galvanic skin response (GSR) sensor, a plethysmography (PPG) sensor, a skin temperature sensor, a three-axis accelerometer and an electroencephalograph (EEG). It is understood that other combinations of sensor may also be envisaged and is within the scope of the disclosure.

According to some embodiments, the at least three parameters may be selected from heart rate/pulse rate (HR/PR), heart rate variability (HRV) monitor, amplitude of photoplethysmogram, skin conductance level (SCL), number of skin conductance fluctuations (NSCF), blood pressure, movement and any combination thereof.

According to some embodiments, the at least three physiological parameters may at least include heart rate (HR), heart rate variability (HRV) and amplitude of photoplethysmogram. According to some embodiments, the at least three physiological parameters may at least include heart rate variability (HRV), amplitude of photoplethysmogram and skin conductance level (SCL). According to some embodiments, the at least three physiological parameters may at least include amplitude of photoplethysmogram, skin conductance level (SCL) and arterial blood pressure. According to some embodiments, the at least three physiological parameters may at least include heart rate variability (HRV), amplitude of photoplethysmogram, and movement. According to some embodiments, the at least three physiological parameters may at least include heart rate variability (HRV), amplitude of photoplethysmogram, skin conductance level (SCL), number of skin conductance fluctuations (NSCF). It is understood that other parameters and combination of parameters, such as any combination of the parameters and features disclosed in table 1 herein, may also be envisaged and are within the scope of the present disclosure.

According to some embodiments, the NS value may enable differentiation between no nociception, and nociception with a sensitivity of above 70% at a specificity of at least 70%. According to some embodiments, the NS value may enable differentiation between no nociception, and nociception with a sensitivity of above 70% at a specificity of at least 75%. According to some embodiments, the NS value may enable differentiation between no nociception, and nociception with a sensitivity of above 75% at a specificity of at least 75%. According to some embodiments, NS value may enable differentiation between no nociception and nociception with a sensitivity of above 80% at a specificity of at least 75%. According to some embodiments, the NS value may enable differentiation between no nociception, and nociception with a sensitivity of above 85% at a specificity of at least 75%. According to some embodiments, the NS value may enable differentiation between no nociception and nociception with a sensitivity of above 70% at a specificity of at least 84%. According to some embodiments, the NS value may enable differentiation between no nociception and nociception with a sensitivity of above 80% at a specificity of at least 84%. According to some embodiments, the NS value may enable differentiation between no nociception and nociception with a sensitivity of above 85% at a specificity of at least 84%. According to some embodiments, the NS value may enable differentiation between no nociception and mild nociception with a sensitivity of above 75% at a specificity of at least 75%.

According to some embodiments, there is provided a medical monitoring device including at least one sensor adapted to sense at least three physiological parameters of a patient administered with an analgesic, and a computing unit adapted to receive the at least three physiological parameters from the at least one sensor, to compute a nociception scale (NS) value based on an analysis of the at least three physiological parameters and/or features derived therefrom, and to determine an amount of an analgesic required to reduce the NS value below a predetermined threshold value. According to some embodiments, the computing unit may be adapted to receive the at least three physiological parameters from the at least one sensor and to compute a nociception scale (NS) value based on an integrative analysis of the at least three physiological parameters and/or features derived therefrom and to determine an amount of an analgesic required to reduce the NS value below a predetermined threshold value.

According to some embodiments, the patient is administered with an analgesic.

According to some embodiments, the NS value is essentially unaffected by vasodilating and/or bradycardial effects of analgesics and/or anesthetics.

According to some embodiments, the at least one sensor is selected from bio-potential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a us blood pressure sensor, a three-axis accelerometer, a diffused correlation spectroscopy (DCS) sensor, an acoustics sensor, a bio-impedance sensor and a piezoelectric sensor, an audio sensor, a motion sensing input device or any combination thereof.

According to some embodiments, the device includes at least three sensors. According to some embodiments the at least three sensors comprise a PPG sensor, a GSR sensor and a three-axis accelerometer and/or a skin temperature sensor. According to some embodiments, the at least three sensors may include at least a plethysmography (PPG) sensor, a skin temperature sensor and a three-axis accelerometer. According to some embodiments, the at least three sensors may include at least galvanic skin response (GSR) sensor, a skin temperature sensor and a three-axis accelerometer. According to some embodiments, the at least three sensors may include at least a galvanic skin response (GSR) sensor, a plethysmography (PPG) sensor and an electrocardiograph (ECG). According to some embodiments, the at least three sensors may include at least a galvanic skin response (GSR) sensor, a plethysmography (PPG) sensor and an electroencephalograph (EEG). According to some embodiments, the at least three sensors may include at least a galvanic skin response (GSR) sensor, a plethysmography (PPG) sensor and an electrooculograph (EOG). According to some embodiments, the at least three sensors may include at least an electrocardiograph (ECG), a skin temperature sensor and a three-axis accelerometer. According to some embodiments, the at least three sensors may include at least a galvanic skin response (GSR) sensor, a plethysmography (PPG) sensor and a facial electromyograph (FEMG). According to some embodiments, the at least three sensors may include at least an electrocardiograph (ECG), a plethysmography (PPG) sensor and an electroencephalograph (EEG). According to some embodiments, the at least three sensors may include at least a galvanic skin response (GSR) sensor, a plethysmography (PPG) sensor and respiration sensor. According to some embodiments, the at least three sensors may include a galvanic skin response (GSR) sensor, a plethysmography (PPG) sensor, a skin temperature sensor, a three-axis accelerometer and an electroencephalograph (EEG). It is understood that other combinations of sensor may also be envisaged and is within the scope of the disclosure.

According to some embodiments, the at least three physiological parameters may be selected from heart rate (HR), pulse rate (PR), heart rate variability (HRV), amplitude of photoplethysmogram, skin conductance level (SCL), number of skin conductance fluctuations (NSCF), arterial blood pressure, movement and any combination thereof. According to some embodiments, the at least three physiological parameters may at least include heart rate (HR), heart rate variability (HRV) and amplitude of photoplethysmogram. According to some embodiments, the at least three physiological parameters may at least include heart rate variability (HRV), amplitude of photoplethysmogram and skin conductance level (SCL). According to some embodiments, the at least three physiological parameters may at least include amplitude of photoplethysmogram, skin conductance level (SCL) and arterial blood pressure. According to some embodiments, the at least three physiological parameters may at least include heart rate variability (HRV), amplitude of photoplethysmogram, and movement. According to some embodiments, the at least three physiological parameters may at least include heart rate variability (HRV), amplitude of photoplethysmogram, skin conductance level (SCL), number of skin conductance fluctuations (NSCF). It is understood that other parameters and combination of parameters, such as any combination of the parameters and features disclosed in table 1 herein, may also be envisaged and are within the scope of the present disclosure.

According to some embodiments, there is provided a device for determining analgesic efficacy including at least one sensor adapted to sense at least three physiological parameters of a patient before (A0) and after (A1) administration of an analgesics and before (S0) and after (S1) providing a noxious stimuli; and a computing unit adapted to receive the at least three physiological parameters from the at least one sensor obtained for A0, A1, S0, S1 and/or combinations thereof, to compute nociception scale (NS) values for A0, A1, S0, S1 and/or combinations thereof based on an analysis of the at least three physiological parameters and/or features derived therefrom, and to determine the efficacy of the analgesic based on a comparison of the nociception scale (NS) values obtained at A0, A1, S0, S1 and/or combinations thereof. According to some embodiments, the computing unit may be adapted to receive the at least three physiological parameters from the at least one sensor obtained for A0, A1, S0, S1 and/or combinations thereof, to compute nociception scale (NS) values for A0, A1, S0, S1 and/or combinations thereof based on an integrative analysis of the at least three physiological parameters and/or features derived therefrom, and to determine the efficacy of the analgesic based on a comparison of the nociception scale (NS) values obtained at A0, A1, S0, S1 and/or combinations thereof.

According to some embodiments, determining the efficacy of the analgesics may include determining an efficient dose of the analgesics.

According to some embodiments, the NS value is essentially unaffected by vasodilating and/or bradycardial effects of analgesics and/or anesthetics.

According to some embodiments, the at least one sensor is selected from bio-potential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a us blood pressure sensor, a three-axis accelerometer, a diffused correlation spectroscopy (DCS) sensor, an acoustics sensor, a bio-impedance sensor and a piezoelectric sensor, an audio sensor, motion sensing input device or any combination thereof.

According to some embodiments, the device may include at least three sensors. According to some embodiments, at least three sensors comprise a PPG sensor, a GSR sensor and a three-axis accelerometer and/or a skin temperature sensor. According to some embodiments, the at least three sensors may include at least a plethysmography (PPG) sensor, a skin temperature sensor and a three-axis accelerometer. According to some embodiments, the at least three sensors may include at least a galvanic skin response (GSR) sensor, a skin temperature sensor and a three-axis accelerometer. According to some embodiments, the at least three sensors may include at least a galvanic skin response (GSR) sensor, a plethysmography (PPG) sensor and an electrocardiograph (ECG). According to some embodiments, the at least three sensors may include at least a galvanic skin response (GSR) sensor, a plethysmography (PPG) sensor and an electrooculograph (EOG). According to some embodiments, the at least three sensors may include at least an electrocardiograph (ECG), a skin temperature sensor and a three-axis accelerometer. According to some embodiments, the at least three sensors may include at least a galvanic skin response (GSR) sensor, a plethysmography (PPG) sensor and a facial electromyograph (FEMG)/electroencephalograph (EEG). According to some embodiments, the at least three sensors may include at least an electrocardiograph (ECG), a plethysmography (PPG) sensor and a facial electromyograph (FEMG)/electroencephalograph (EEG). According to some embodiments, the at least three sensors may include at least a galvanic skin response (GSR) sensor, a plethysmography (PPG) sensor and respiration sensor. It is understood that other combinations of sensor may also be envisaged and is within the scope of the disclosure.

According to some embodiments, the at least three physiological parameters are selected from heart rate (HR), pulse rate (PR), heart rate variability (HRV), amplitude of photoplethysmogram, skin conductance level (SCL), number of skin conductance fluctuations (NSCF), arterial blood pressure, movement and any combination thereof. According to some embodiments, the at least three physiological parameters may at least include heart rate (HR), heart rate variability (HRV) and amplitude of photoplethysmogram. According to some embodiments, the at least three physiological parameters may at least include heart rate variability (HRV), amplitude of photoplethysmogram and skin conductance level (SCL). According to some embodiments, the at least three physiological parameters may at least include amplitude of photoplethysmogram, skin conductance level (SCL) and arterial blood pressure. According to some embodiments, the at least three physiological parameters may at least include heart rate variability (HRV), amplitude of photoplethysmogram, and movement. According to some embodiments, the at least three physiological parameters may at least include heart rate variability (HRV), amplitude of photoplethysmogram, skin conductance level (SCL), number of skin conductance fluctuations (NSCF). It is understood that other parameters and combination of parameters, such as any combination of the parameters and features disclosed in table 1 herein, may also be envisaged and are within the scope of the present disclosure.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIG. 1A schematically illustrates a nociception/analgesia monitoring device, according to some embodiments.

FIG. 1B schematically illustrates a nociception/analgesia monitoring device, according to some embodiments.

FIG. 2 is an illustrative flowchart of a method for determining a nociception scale (NS) value, according to some embodiments.

FIG. 3 is an illustrative flowchart of a method for determining an optimal dose of an analgesic, according to some embodiments.

FIG. 4 is an illustrative flowchart of a method for analgesic efficacy, according to some embodiments.

FIG. 5 is an illustrative flowchart of a method for determining the amount of an analgesic required to maintain nociception levels within a desired target range.

FIG. 6A shows bispectral index before (open symbol) and after (closed symbol) noxious stimulation, *paired t-test p<0.001; # unpaired t-test p<0.001.

FIG. 6B shows heart rate (HR) before (open symbol) and after (closed symbol) noxious stimulation, *paired t-test p<0.001; # unpaired t-test p<0.001.

FIG. 6C shows mean arterial pressure (MAP) before (open symbol) and after (closed symbol) noxious stimulation, *paired t-test p<0.001; # unpaired t-test p<0.001.

FIG. 6D shows nociception scale (NS) before (open symbol) and after (closed symbol) noxious stimulation, *paired t-test p<0.001; # unpaired t-test p<0.001.

FIG. 7 shows discrimination between nociceptive stimuli (incision and intubation) and non-nociceptive period: Receiver operating characteristic (ROC) curves of the HR, MAP and NS values.

FIG. 8 shows ROC curves of the ΔHR, ΔMAP and ΔNS signals.

FIG. 9A shows a boxplot of the effect of remifentanil on NS before noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 9B shows a boxplot of the effect of remifentanil on NS after noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 9C shows a boxplot of the effect of remifentanil on NS before noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 9D shows a boxplot of the effect of remifentanil on NS after noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10A shows shows a boxplot of the effect of remifentanil on heart rate (HR) before noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10B shows a boxplot of the effect of remifentanil on heart rate (HR) after noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10C shows a boxplot of the effect of remifentanil on heart rate (HR) before noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10D shows a boxplot of the effect of remifentanil on heart rate (HR) after noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 11A shows shows a boxplot of the effect of remifentanil on mean arterial pressure (MAP) before noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

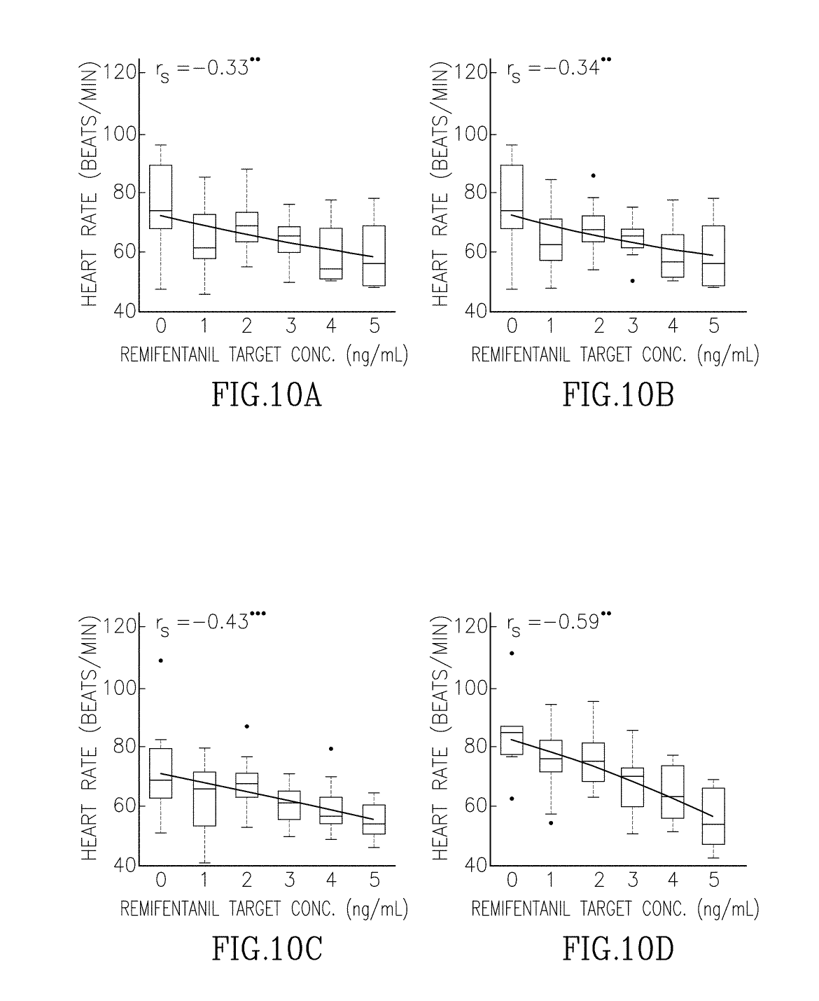

FIG. 10B shows a boxplot of the effect of remifentanil on mean arterial pressure (MAP) after noxious stimulation under non-nociceptive conditions. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10C shows a boxplot of the effect of remifentanil on mean arterial pressure (MAP) before noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 10D shows a boxplot of the effect of remifentanil on mean arterial pressure (MAP) after noxious stimulation after intubation. Boxplots represent the median 25^{th} and 75^{th} percentile, the whiskers extend to the most extreme data points; outliers are plotted individually (block dots). The Spearman correlation is given (rS), with * indicating p<0.05 and *** p<0.001. A quadratic polynomial is fitted to the data to guide the eye.

FIG. 12 shows ROC analysis for discrimination of noxious stimuli (TP1+TP2) from non-noxious period (TNP). Dashed lines-reaction (A) values; Solid lines- post values. HR & PPGA reaction values are normalized (norm).

FIG. 13 shows the response to clinical stimuli TP1, TP2, TNP by post and reaction (A) values. For each box: central mark=median; edges of box=25-75%; whiskers extend to the most extreme data points; outliers are plotted individually by +. * p<0.00625, ** p<0.001, *** p<0.0001.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to monitoring nociception/analgesia, specifically to nociception/analgesia monitoring during anesthesia and pain management.

According to some embodiments, there is provided a nociception/analgesia monitoring device configured to assess the nociception level of a patient.

According to some embodiments, the patient is administered with and/or under influence of an anesthetic and/or an analgesic. According to some embodiments, the assessment of the patient's nociception level is essentially unaffected by vasodilating and/or bradycardial effects caused by the drugs administered to the patient.

According to some embodiments, the assessment of the patient's nociception level includes identifying and/or distinguishing between mild nociception and severe nociception. According to some embodiments, the assessment of the patient's nociception level includes identifying and/or distinguishing between mild nociception, moderate nociception and/or severe nociception.

As referred to herein, the terms “patient” may relate to a subject undergoing nociception/analgesia monitoring. According to some embodiments, the patient may refer to a subject undergoing surgery or other potentially pain inducing medical intervention. According to some embodiments, the patient may refer to a subject administered with an anesthetic and/or an analgesic.

As used herein, the terms “drug” and “medicament” may be interchangeable and may refer to any medication administered to the patient. According to some embodiments, the drug is an anesthetic and/or analgesic. According to some embodiments, the drug is an opioid. According to some embodiments, the drug is a muscle relaxant. According to some embodiments, the drug is a beta-blocker. According to some embodiments, the drug is selected from remifentanil, fentanyl, propofol, isoflurane, desflutane, sevoflurane, halotane, ketamine, thiopental, rocuronium, lidocaine, atracurium, rapacuronium, NSAIDs, Nitros-Oxide. Each possibility is a separate embodiment.

According to some embodiments, the term “nociception” may refer to the encoding and processing of harmful stimuli in the nervous system, including physiological responses to surgical and other clinical noxious stimuli during unconsciousness. According to some embodiments, the terms “nociception” and “pain” may be used interchangeably. According to some embodiments, nociception may refer to a physiologic process and pain as a subjective phenomenon.

As used herein, the term “analgesia” may refer to the relief or reduction in pain perception, including the reduction or absence of a pain perception during a painful stimulus, e.g. pain caused during surgery. According to some embodiments, the terms “pain monitoring”, “nociception monitoring”, “analgesia monitoring” and “pain assessment” may be interchangeably used.

According to some embodiments, the term “nociception level” may refer to a continuous and/or incremental scale of pain ranging from no nociception to severe nociception (for example, but not limited to, a numerical scale from 0-10 or from 0-100). Additionally or alternatively, a nociception level may refer to a category of nociception, such as no nociception, mild nociception, severe nociception or any other suitable category defining a level of nociception.

According to some embodiments, the term “no nociception” refers to a patient presenting no physiological signals and/or neural processes indicative of nociception. According to some embodiments, as used herein no nociception may refer to a nociception scale value in the range of, for example, 0-20 out of a 0-100 numerical scale.

According to some embodiments, the term “mild nociception” refers to patients presenting mild deviations in physiological signals and/or neural processes, as compared to no nociception. According to some embodiments, as used herein, mild nociception may refer to a nociception scale value in the range of, for example, 20-50 out of a 0-100 numerical scale.

According to some embodiments, the term “moderate nociception” refers to patients presenting moderate deviations in physiological signals and/or neural processes, as compared to no nociception. According to some embodiments, as used herein, mild nociception may refer to a nociception scale value in the range of, for example, 40-70 out of a 0-100 numerical scale.

According to some embodiments, the term “severe nociception” refers to patients presenting pronounced deviations in physiological signals and/or neural processes, as compared to no nociception. According to some embodiments, as used herein, severe nociception may refer to a nociception scale value in the range of, for example, 60-100 out of a 0-100 numerical scale.

According to some embodiments, the monitoring device may include at least one sensor. As used herein the term “at least one sensor” may refer to 1, 2, 3, 4, 5, 6 or more sensors. Each possibility is a separate embodiment. According to some embodiments, the at least one sensor may be incorporated into a finger probe, a wristwatch, a wristband, a glove, a chest band or any other suitable element suitable for attachment to the subject, for example, to the subject's finger, wrist or chest.

According to some embodiments, the at least one sensor is selected from a bio-potential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a blood pressure sensor, an accelerometer, a diffused correlation spectroscopy (DCS) sensor, an acoustics sensor, a bio-impedance sensor, a piezoelectric sensor, an audio sensor, motion sensing input device (e.g. an image/video recorder and/or analyzer) configured to, in conjunction with suitable software, identify body or facial spasms and or twitches, or any combination thereof. Each possibility is a separate embodiment.

According to some embodiments, the monitoring device may include at least a PPG sensor and a GSR sensor. According to some embodiments, the monitoring device may include at least a PPG sensor, a GSR sensor and a three-axis accelerometer. According to some embodiments, the monitoring device may include at least a PPG sensor, a GSR sensor and a skin temperature sensor. According to some embodiments, the monitoring device may include at least a PPG sensor, a GSR sensor, a skin temperature sensor and a three-axis accelerometer. According to some embodiments, the monitoring device may include at least a plethysmography (PPG) sensor, a skin temperature sensor and a three-axis accelerometer. According to some embodiments, the monitoring device may include at least a galvanic skin response (GSR) sensor, a skin temperature sensor and a three-axis accelerometer. According to some embodiments, the at least three sensors may include at least a galvanic skin response (GSR) sensor, a plethysmography (PPG) sensor and an electrocardiograph (ECG). According to some embodiments, the at least three sensors may include at least a galvanic skin response (GSR) sensor, a plethysmography (PPG) sensor and an electrooculograph (EOG). According to some embodiments, the at least three sensors may include at least an electrocardiograph (ECG), a skin temperature sensor and a three-axis accelerometer. According to some embodiments, the at least three sensors may include at least a galvanic skin response (GSR) sensor, a plethysmography (PPG) sensor and a facial electromyograph (FEMG)/electroencephalograph (EEG). According to some embodiments, the at least three sensors may include at least an electrocardiograph (ECG), a plethysmography (PPG) sensor and a facial electromyograph (FEMG)/electroencephalograph (EEG). According to some embodiments, the at least three sensors may include at least a galvanic skin response (GSR) sensor, a plethysmography (PPG) sensor and respiration sensor. It is understood that other combinations of sensor may also be envisaged and is within the scope of the disclosure.

According to some embodiments, the at least one sensor may be configured to sense at least three physiological parameters of the patient. As used herein the term “at least three physiological parameters” may refer to 3, 4, 5, 6 or more physiological parameters. Each possibility is a separate embodiment. However, according to some alternative embodiments, less than three parameters may be sensed, such as one or two parameters. According to some embodiments, the at least one sensor may be configured to sense a plurality of physiological parameters of the patient. As used herein the term “plurality” with regards to physiological parameters may refer to more than 5 physiological parameters, more than 10 physiological parameters or any other suitable number of parameters. Each possibility is a separate embodiment. As a non-limiting example, the monitoring device may include two sensors configured to sense at least three physiological parameters.

According to some embodiments, the at least three physiological parameters are indicative of a nociception level of the patient. According to some embodiments, the at least three physiological parameters may be obtained from a same sensor and/or from different sensors. According to some embodiments, the at least three physiological parameters may be selected from blood pressure, respiration, internal and/or surface temperature, pupil diameter, galvanic skin response, and signals received and/or extracted and/or derived from ECG, PPG, EOG, EGG, EEG, EMG, LDV, capnograph, accelerometer, audio, image and/or video identifying body or facial spasms and or twitches or any combination thereof. Each possibility is a separate embodiment.

According to some embodiments, the at least three physiological parameters may be selected from heart rate/pulse rate (HR/PR)), heart rate variability (HRV) monitor, amplitude of photoplethysmogram (PPGA), skin conductance level (SCL), number of skin conductance fluctuations (NSCF), blood pressure and movement. Each possibility is a separate embodiment. According to some embodiments, the at least three physiological parameters may at least include heart rate variability (HRV), amplitude of photoplethysmogram and movement. According to some embodiments, the at least three physiological parameters may at least include heart rate variability (HRV), arterial blood pressure and number of skin conductance fluctuations (NSCF). According to some embodiments, the at least three physiological parameters may at least include amplitude of photoplethysmogram, skin conductance level (SCL) and arterial blood pressure. According to some embodiments, the at least three physiological parameters may at least include heart rate, amplitude of photoplethysmogram, and movement. According to some embodiments, the at least three physiological parameters may at least include skin conductance level (SCL), EMG Spectral Entropy and arterial blood pressure. According to some embodiments, the at least three physiological parameters may at least include heart rate variability (HRV) monitor, amplitude of photoplethysmogram, skin conductance level (SCL) and number of skin conductance fluctuations (NSCF). According to some embodiments, the at least three physiological parameters may at least include heart rate (HR), amplitude of photoplethysmogram and respiration rate. According to some embodiments, the at least three physiological parameters may at least include skin conductance level (SCL), EEG coherence, amplitude of photoplethysmogram. According to some embodiments, the at least three physiological parameters may at least include skin conductance level (SCL), EEG Spectral Entropy and movement. It is understood that other combinations of parameters and combination of parameters may also be envisaged and are within the scope of the present disclosure. According to some embodiments, the at least three physiological parameters may refer to a derivative and/or features of the parameter, a moving average of the parameter, a value of the parameter or its derivative obtained during a moving window, derivatives of the parameter in time and the like. According to some embodiments, a derivative of the parameter may refer to any value derived from the parameter by filtering, averaging, and/or other mathematical or statistical processing.

According to some embodiments, the at least three physiological parameters include features derived from physiological signals obtained from the sensors. As used herein, the terms “features”, “physiological features” and “extracted features” may be interchangeably used and may refer to at least one or more physiological features that may be extracted and/or derived from sensor signals. The features may be quantitative or qualitative.

According to some embodiments, a plurality of features may be derived from the physiological parameters of the patient. As used herein the term “plurality” with regards to features may refer to more than 5 physiological parameters, more than 10 physiological parameters, more than 15 physiological parameters or any other suitable number of parameters. Each possibility is a separate embodiment.

According to some embodiments, the features may be derived using feature extraction techniques and may include combining a plurality of extracted features, for example, by non-linear regression techniques.

Within the context of the present invention the terms “feature extraction”, “feature processing” and “signal processing” may refer to the processes, manipulations and signal processing measures performed to analyze a physiological parameter. Non-limiting examples of suitable physiological features are depicted in table 1, below.

TABLE 1
physiological features

| Number | | | |
|---|---|---|---|
| of | | | |
| features | Signal | Feature | Description |
| 15 | ECG | Q/R/S/T/P | Amplitude, moving average amplitude and variability of |
| | | amplitude, | amplitude of the Q/R/S/T/P pulse-an array that represent |
| | | average and | the location and the amplitude of the peak |
| | | variability | |
| 15 | ECG | RR/PQ/PR/ | The interval, moving average of interval and variability |
| | | QT/RS | of interval between each pulse or between internal pulse |
| | | interval, | waves, an array that represent the location of the value |
| | | average and | computed, a sthe first peak location and its relevant |
| | | variability | interval |
| 1 | ECG | P wave | Width of the P wave-an array that represent the location |
| | | width | of the P peak and P wave relevant width |
| 1 | ECG | ST level | The point of inflection after S wave, which defines |
| | | | beginning of ST segment. An array that represent the |
| | | | location and point amplitude. |
| 5 | ECG | Q, R, S, T, P | Derivative of the Amplitude |
| | | amplitude | |
| | | change | |
| 5 | ECG | RR/PQ/PR/ | Derivative of the pulses intervals |
| | | QT/RS | |
| | | interval | |
| | | change | |
| 1 | ECG | ST level | Derivative of the ST level |
| | | change | |
| 1 | ECG | QRS width | Derivatives of width of the QRS complex |
| | | change | |
| 1 | ECG | Energy of | Computing the energy of the residues after applying the |
| | | ECG | spectral cleaning and after applying auto regressive |
| | | residues | methods |
| 1 | ECG | Number of | Number of missing R-peaks for a certain time window |
| | | missing R | |
| | | peaks | |
| 4 | ECG | R-R | Power (area) of the VLF, LF, MF and HF frequencies |
| | Freq. | Variability | analysis of the interval variability between each pulse in a |
| | | VLF, LF, MF | given resolution as was defined above in Heart Rate |
| | | and HF | variability paragraph |
| 1 | ECG | R-R | Ratio between LF HRV power and HF HRV power |
| | Freq. | Variability | |
| | | LF/HF | |
| 1 | ECG | RRI | Wavelet analysis of the interval variability between each |
| | Freq. | Variability | pulse in a given resolution. |
| | | wavelet | |
| | | analysis | |
| 1 | ECG | alpha | Slope of HRV power spectrum |
| | Freq. | | |
| 1 | ECG | beta | Slope of the log of HRV PS |
| | Freq. | | |
| 3 | Respi- | Upper peak | The peaks value, moving average of interval and |
| | ratory | values, | variability of peaks amplitude. The peak represents the |
| | | average, | depth of respiration how deep we take a breath. |
| | | variability | |
| 3 | Respi- | Lower Peak | The lower peaks value, moving average of interval and |
| | ratory | values, | variability of peaks amplitude. The peaks represent the |
| | | average, | depth of breath release. |
| | | variability | |
| 3 | Respi- | Respiratory | The rate is 1/Peak to peak distance. The interval rate, |
| | ratory | rate, average | average rate and variability of the rate |
| | | and variability | |
| 1 | Respi- | Spectrum | Spectrum analysis of the respiratory signal |
| | ratory | Analysis of | |
| | | the respiratory | |
| 1 | Respi- | Power (area) | The area bellow the breath signal |
| | ratory | | |
| 6 | PPG | PPG Peak | An array that represents location and amplitude of Peak |
| | | and Trough | and Trough. Peak denotes a point of maximum blood |
| | | Amplitude, | volume in a finger; Trough denotes a minimum basal blood |
| | | average and | volume. Both amplitude, moving average of the amplitude |
| | | variability | and variability are calculated |
| 1 | PPG | PPG | An array that represents location and amplitude of a point |
| | | maximum | between onset injection and Peak where maximum rate of |
| | | rate point | blood volume increase is observed |
| 1 | PPG | PPG dicrotic | An array that represents location and amplitude of PPG |
| | | notch | dicrotic notch. |
| 12 | PPG | PP/TT/NN/ | Peak to peak, trough to trough, notch to notch, maximum |
| | | MM/ | rate to maximum rate, and other time intervals between |
| | | intervals, | points of interest in PPG beat. Both interval, moving |
| | | average and | average of the interval and variability are calculated-all |
| | | variability | representing the pulse rate |
| 12 | PPG | /PT/PN/NT/ | peak to trough, peak to notch, notch to trough, notch to |
| | | NM intervals, | maximum rate, and other time intervals between points of |
| | | average and | interest in PPG beat. Both interval, moving average of the |
| | | variability | interval and variability are calculated |
| 5 | PPG | PP spectral | Spectrum analysis of the Peak to Peak variability: HF, MF, |
| | | analysis | LF and VLF bands power, LF/HF ration |
| 1 | PPG | Area Under | An array that represents location and integral of single beat |
| | | Curve | of PPG signal (AUC) |
| 1 | PPG | PPG | Time analysis of the envelope of PPG signal. |
| | | envelope-time | (envelope-Peak-Trough of PPG signal) |
| | | analysis | |
| 1 | PPG | PPG | Spectral analysis of the envelope of PPG signal, |
| | | envelope- | (envelope-Peak-Trough of PPG signal) |
| | | spectral | |
| 1 | PPG | PPG | Wavelet analysis of the interval variability between each |
| | | Variability | pulse in a given resolution. |
| | | wavelet | |
| | | analysis | |
| 1 | ECG- | Respiratory | Correlation between the Respiration and the decrease/ |
| | Resp | sinus | increase in R-R interval |
| | | arrhythmia | |
| 1 | PPG- | Respiratory | Correlation between the Respiration and the decrease/ |
| | Resp | sinus | increase in PPG intervals |
| | | arrhythmia | |
| 1 | ECG- | Pulse | An array that represent the location and the delay |
| | BP | Transition | between R peak of ECG signal and Peak of Blood Pressure |
| | | time | signal. (PTT or rPTT) (Weiss, et al. 1980) |
| 1 | ECG- | Pulse | An array that represents the location and the delay |
| | PPG | Transition | between R peak of ECG signal and Peak of PPG |
| | | time | signal (PTT or rPTT). |
| 1 | PPG- | Pulse | An array that represents the location and the delay between |
| | PPG | Transition | two PPG signals located on the same arteriole in different |
| | | time | (PTT or rPTT). |
| 1 | CNIBP | Average/ | Average and variability (moving average and moving |
| | | variability | variability) |
| | | mean aortic | |
| | | pressure | |
| | | (Pmean) | |
| 6 | CNIBP | CBP Peak, | An array that represents location and amplitude of Peak |
| | | and Trough | and Trough. Peak denotes the systolic BP; Trough denotes |
| | | amplitude, | the diastolic. Amplitude, moving average amplitude and |
| | | average and | variability are calculated |
| | | variability | |
| 1 | CNIBP | Blood onset | An array that represents location and amplitude of a point |
| | | ejection point | after Trough where blood ejection is started (maximum |
| | | | second derivative) |
| 1 | CNIBP | CBP | An array that represents location and amplitude of a point |
| | | maximum | between onset injection and Peak where maximum rate of |
| | | rate point | blood volume increase is observed (middle of Anacrotic |
| | | | rise) |
| 1 | CNIBP | CBP dicrotic | An array that represents location and amplitude of CBP |
| | | notch | dicrotic notch. |
| 15 | CNIBP | PP/PT/PN/ | Peak to peak, peak to trough, peak to notch, notch to trough, |
| | | NT/NM | notch to maximum rate, and other time intervals between |
| | | intervals, | points of interest in BP beat. |
| | | average and | |
| | | variability | |
| 1 | CNIBP | PP spectral | Spectrum analysis of the Peak to Peak variability: HF, MF, |
| | | analysis | LF and VLF bands power, LF/HF ration |
| 1 | CNIBP | Area Under | An array that represents location and integral of single beat |
| | | Curve | of PPG signal (AUC) |
| 1 | CNIBP | BP variability | Wavelet analysis of the interval variability between each |
| | | wavelet | pulse in a given resolution. |
| | | analysis | |
| 2 | GSR | Average/ | Average and variability of perspiration (moving average |
| | | variability | and moving variability) |
| | | Perspiration | |
| 1 | GSR | Peak Interval, | The time interval between peaks, the moving average of the |
| | | average and | interval and the variability |
| | | variability | |
| 1 | GSR | Peak | Amplitude, moving average amplitude and variability of |
| | | amplitude, | amplitude of the GSR peaks comparing to the base band |
| | | average, | |
| | | variability | |
| 1 | GSR | General area | The area under each peak |
| 1 | GSR | Phasic EDA, | The first derivative of the GSR signal (EDA phasic), the |
| | | amplitude | moving average of the slopes (normal and absolute values)- |
| | | average and | mean phasic, and the variability of the slopes |
| | | variability | |
| 1 | GSR | spontaneous | The average number of spontaneous fluctuations (SF) in |
| | | fluctuations | an individual |
| | | Count | |
| 1 | GSR | Spectral | The amplitude of the highest peak in the spectrum analysis |
| | | Analysis: Peak | |
| | | Amplitude | |
| 1 | GSR | Spectral | The frequency of the highest peak in the spectrum analysis |
| | | Analysis: Peak | |
| | | Frequency | |
| 1 | GSR | Spectral | The power (integration of signal) in the different frequency |
| | | Analysis: | and specifically in 0.01-0.04 Hz |
| | | Power | |
| 1 | GSR | difference | The differences between the values of the highest peaks in |
| | | between Peak | the spectrum analysis of two different locations |
| | | Amplitude | |
| 1 | GSR | GSR wavelet | Wavelet analysis of the interval variability between each |
| | | analysis | pulse in a given resolution. |
| 2 | Temper- | Average/ | Average and variability of perspiration (moving average |
| | ature | variability | and moving variability) |
| | | Temperature | |
| 1 | Temper- | Peak Interval, | The time interval between peaks, the moving average of |
| | ature | average and | the interval and the variability |
| | | variability | |
| 1 | Temper- | Peak | Amplitude, moving average amplitude and variability of |
| | ature | amplitude, | amplitude of the temperature peaks comparing to the base |
| | | average, | band |
| | | variability | |
| 1 | Temper- | derivative | The first derivative of the temperature signal, the moving |
| | ature | amplitude | average of the slopes (normal and absolute values) and the |
| | | average and | variability of the slopes |
| | | variability | |
| 1 | Temper- | Spectral | The value of the highest peak in the spectrum analysis |
| | ature | Analysis: Peak | |
| | | Amplitude | |
| 1 | Temper- | Spectral | The frequency of the highest peak in the spectrum analysis |
| | ature | Analysis: Peak | |
| | | Location | |
| 1 | Temper- | Spectral | The power (integration of signal) in 0.01-0.04 Hz |
| | ature | Analysis | |
| | | Power | |
| 1 | Temper- | Temperature | Wavelet analysis of the interval variability between each |
| | ature | wavelet | pulse in a given resolution. |
| | | analysis | |
| 2 | EOG | Average/ | |
| | | variability | |
| 4 | EEG/ | A, β, γ, δ, θ | Classical EEG frequency band definitions. |
| | EMG | ratio between | Frequency band Frequency range [Hz] |
| | | the powers | delta, δ 0.5-4-deep sleep (Sometimes is referred as 1-3.5) |
| | | | theta, θ 4-8-drowsiness (Sometimes is referred as 3.5-8) |
| | | | alpha, α 8-14-relaxed but alert (sometimes is referred as |
| | | | 8-13) |
| | | | beta, β 14-30-highly alert and focused (sometimes is |
| | | | referred as 13-30) |
| | | | gamma γ, 30-70-represent binding of different populations |
| | | | of neurons together into a network for the purpose of |
| | | | carrying out a certain cognitive or motor function |
| | | | (sometimes is referred as 36-100) |
| 1 | EEG/ | Average/ | |
| | EMG | variability | |
| 1 | EEG/ | median/ | The frequency at which the median power is reached |
| | EMG | frequency | |
| 1 | EEG/ | mean/ | The frequency at which the average power is reached |
| | EMG | frequency | |
| 1 | EEG/ | Mean power | The average power of the spectrum within epoch |
| | EMG | | |
| 1 | EEG/ | Peak | The frequency at which the power reaches its peak |
| | EMG | frequency | |
| 1 | EEG/ | Spectral Edge | The spontaneous EEG frequency below which x percent |
| | EMG | Frequency | of the power are located. Typically x is in the range 75 to |
| | | | 95. SEF has variously been used to estimate the depth of |
| | | | anesthesia. |
| 1 | EEG/ | Approximate | For details see (Bruhn, Ropcke and Hoeft 2000) |
| | EMG | Entropy- | |
| 1 | EEG/ | BSR-Burst | The burst suppression ratio is the proportion of the |
| | EMG | Suppression | suppression EEG in the analyzed epoch (usually one |
| | | ratio | minute): |
| | | | $BSR = \frac{total of suppression}{epoch length} 100 \%$ |
| 1 | EEG/ EMG | BcSEF | Burst compensated spectral edge frequency $BcSEF = SEF ( 1 - \frac{BSR \%}{100 \%} )$ |
| 1 | EEG/ | WSMF | A generalized form of spectral edge frequency, referred to |
| | EMG | | as weighted spectral median frequency (WSMF), edge |
| | | | frequency is calculated not necessarily from PSD but from |
| | | | amplitude spectrum, which is raised to the power p = |
| | | | [0.1 . . . 2.4]; second, the cutoff frequencies of the original |
| | | | spectrum are well-defined; and, third, factor r = |
| | | | [0:05 : : : 0:95] is used, the percentile of the spectrum (e.g., |
| | | | r = 0:5 for MF and r = 0:95 for SEF). |
| 1 | EEG/ | CUP | Canonical univariate parameter: frequency bins with a |
| | EMG | | width of 3 Hz or classical frequency bands are optimally |
| | | | weighted to obtain the best possible correlation with the |
| | | | drugs' effect-site concentration as obtained from |
| | | | pharmacokinetic-pharmacodynamic (PK-PD) |
| | | | $CUP = {\sum }_{k = 1}^{10} {γ }_{k} log {p}_{k} modeling$ |
| 1 | EEG/ EMG | SpEn- | Spectral Entropy $SpEn = - {\sum }_{k}^{N} {p}_{k} log {p}_{k} .$ |
| 1 | EEG/ EMG | BcSpEn- | Burst compensated Spectral Entropy $BcSpEn = SpEn ( 1 - \frac{BSR ( \% )}{100 \%} ) .$ |
| 1 | EEG/ EMG | Beta Ratio | $BetaRatio = log \frac{{\hat{P}}_{30 - 47 Hz}}{{\hat{P}}_{11 - 20 Hz}} .$ |
| 4 | EEG/ | Histogram | Mean, Standard deviation, Kurtosis, Skewness of signal |
| | EMG | parameters | histogram |
| N | EEG/ | AR | Parameters of AR representation (Schlogl 2006) |
| | EMG | parameters | |
| 3 | EEG/ | Normalized | NSD parameters can be defined by means of first and |
| | EMG | slope | second derivatives. “Activity” is a measure of the mean |
| | | descriptors | power, “Mobility” is an estimate of the mean frequency and |
| | | (Hjorth | “Complexity” is an estimate of the bandwidth of the signal |
| | | parameters) | (frequency spread) (Hjorth 1973). |
| 3 | EEG/ | Barlow | Parameters based on Barlow EEG model which is an |
| | EMG | parameters | alternative time frequency decomposition. Parameters such |
| | | | as Running Mean Frequency and Spectral Purity Index |
| | | | (Goncharova and Barlow 1990) |
| 3 | EEG/ | Wackermann | Three multi-channel linear descriptors of EEG signal. |
| | EMG | parameters | spatial complexity (Ω), field power (Σ) and frequency of |
| | | | field changes (Φ) (Wackermann 1999) |
| 1 | EEG/ | Brain rate | Weighted Mean Frequency (Pop-Jordanova and |
| | EMG | | Pop-Jordanov 2005) |
| 1 | EEG/ EMG | SynchFastSlow | $SynchFastSlow = log \frac{{\hat{B}}_{40 - 47 Hz}}{{\hat{B}}_{0.5 - 47 Hz}} .$ |
| | | | The spectrum and bispectrum, derived from two-second |
| | | | epochs, are smoothed using a running average against those |
| | | | calculated in the previous minute. 3 minutes window is |
| | | | required to obtain a consistent estimate of the bicoherence. |
| 1 | EEG/ | 80 Hz | Ocular microtremor (OMT) is a constant, physiological, |
| | EMG | frequency | high frequency (peak 80 Hz), low amplitude (estimated |
| | | in EEG near | circa 150-2500 nm) eye tremor. |
| | | the eyes | |
| 3 | EMG | Average/ | Average rectified value (mean of the absolute windowed |
| | | variability/ | signal) |
| | | and entropy | |
| 1 | EMG | Spectrum | Calculate the power of each frequency area-the location |
| | | analysis- | of the EMG should be defined |
| 1 | EMG | median | The frequency at which the median power is reached |
| | | frequency | |
| 1 | EMG | mean | The frequency at which the average power is reached |
| | | frequency | |
| 1 | EMG | Mean | The average power of the spectrum within epoch |
| | | power | |
| 1 | EMG | Peak | The frequency at which the power reaches its peak |
| | | frequency | |
| 1 | EMG | Mean | The average power of the power spectrum within the epoch |
| | | power | |
| 1 | EMG | Total power | The sum of the power spectrum within the epoch |
| 1 | EMG | spontaneous | Lower oesophageal contractility (LOC). The non-striated |
| | | lower | muscles in the lower half of oesophagus retain their |
| | | oesophageal | potential activity even after full skeletal muscle paralysis |
| | | contractions | by neuromuscular blocking agents. Spontaneous lower |
| | | (SLOC) | oesophageal contractions (SLOC) are non-propulsive |
| | | | spontaneous contractions mediated via vagal motor nuclei |
| | | | and reticular activating system in the brain stem. The |
| | | | frequency of these movements is increased as the dose of |
| | | | the anaesthetic is reduced. (Thomas and Evans 1989) |
| 1 | SVmR | Signal | SVmR-skin vasomotor reflexes-using laser Doppler |
| | | analysis | |
| 2 | Airway | Average/ | End tidal Carbon Dioxid (anesthesia) |
| | CO2 | Variability | |
| 1 | Airway | Average | End tidal sevofluane (anesthesia) |
| | Gases | | |
| 2 | Pneumo- | Average/ | PVR-Pulse Volume Recording-Average/Variability |
| | plethys- | Variability | of signal's amplitude and signal analysis |
| | mograph | | |
| N | All | Change | The baseline is computed during the first minutes-when |
| | Signals | from the | the patient is in a constant position reflecting the position |
| | | baseline of | of the treatment, with no pain stimuli. The differences |
| | | this patient | (distance) of the parameters from this values are calculated |
| | | | (see ‘Normalization per patient’) |
| N | All | Change | The difference in the parameter from its previous sample |
| | Signals | from | (subtraction or proportion or similar formula representing |
| | | previous | a change) |
| | | window | |
| N | All | Cross | Cross correlation between all different signals-canonical |
| | Signals | correlation/ | correlation |
| | | Coherence/ | |
| | | canonical | |
| | | correlation | |
| N | All | Signature in | Signature of a predefined period (for example 60 seconds |
| | Signals | time- | of HR, EEG pattern, or other size of defined segment) |
| | | functional | |
| | | features | |
| 12 | accelero- | Average | accelerometer X, Y, Z theta, movement analysis |
| | meter | value, | |
| | X, Y, Z | Variability | |
| | θ | | |
| 1 | Environ- | Value and | |
| | ment | moving | |
| | Temper- | average | |
| | ature | | |

According to some embodiments, the nociception/analgesia monitoring device includes a processor and/or other computing unit. According to some embodiments, the computing unit may be a computing circuity. According to some embodiments, the computing unit may be a remote processing unit such as, but not limited to, a mobile device, smartphone, tablet pc, miniaturized computing device, system on chip, a cloud or the like. According to some embodiments, the nociception/analgesia monitoring device may include an analog to digital (A2D) component. According to some embodiments, the A2D component may be included in the processing unit. According to some embodiments, the A2D component may be included in the (wearable) element attached to the subject, such as the finger probe, the wristwatch, the wristband, the chest band, the glove or the like. According to some embodiments, the A2D component may be included in the sensor. It is understood by one of ordinary skill in the art that inclusion of the A2D component in the wearable element and/or in the sensor may enable a direct transfer of data to remote computing units such as, but not limited to, a cloud. According to some embodiments, the computing unit may be located on or within the finger probe, the wristwatch, the wristband, the chest band, the glove or the like. According to some embodiments, the computing unit may be an external and/or adjunct computing device, such as, but not limited to, a mobile, smartphone, tablet, pc or any dedicated computing device. Each possibility is a separate embodiment. According to some embodiments, the computing unit may be a virtual processor, such as an internet enabled device (i.e. cloud computing).

According to some embodiments, at least part of the computation may be executed by a computation unit incorporated into the finger probe, the wristwatch, the wristband, the chest band, the glove or the like. According to some embodiments, at least part of the computation may be executed by an external and/or adjunct computing device (mobile, smartphone, tablet, pc or any dedicated computing device). According to some embodiments, at least part of the computation may be executed by a virtual processor. According to some embodiments, the entire computation may be executed by a computation unit incorporated into the finger probe. According to some embodiments, the entire computation may be executed by an external and/or adjunct computing device (mobile, smartphone, tablet, pc or any dedicated computing device). According to some embodiments, the entire computation may be executed by a virtual processor. According to some embodiments, the computation may be executed partially by the computation unit incorporated into the finger probe, the wristwatch, the wristband, the chest band, the glove or the like, partially by the adjunct computing device (mobile, smartphone, tablet, pc or any dedicated computing device) and or partially by a virtual processor.

According to some embodiments, the processor may be configured to receive the at least three physiological parameters from the at least one sensor, and to compute a nociception scale (NS) value based on an analysis of the at least three parameters and/or features derived therefrom. According to some embodiments, the processor may be configured to receive the at least three physiological parameters from the at least one sensor, and to compute a nociception scale (NS) value based on an integrative analysis of the at least three parameters and/or features derived therefrom.

According to some embodiments, the term “nociception scale (NS)” may refer to a multidimensional index of nociception obtained through a non-linear combination of nociception-related physiological parameters. According to some embodiments, the NS has been developed to correlate with a reference clinical score of nociception based on calculated opioid concentration and estimated stimulus strength (i.e., the combined index of stimulus and analgesia or CISA).

According to some embodiments, the non-linear regression combination of nociception-related physiological parameters may be made using techniques such as Nearest Shrunken Centroids (NSC), Classification and Regression Trees (CART), ID3, C4.5, Multivariate Additive regression splines (MARS), Multiple additive regression trees (MART), Nearest Centroid (NC), Shrunken Centroid Regularized Linear Discriminate and Analysis (SCRLDA), Random Forest, Boosting, Bagging Classifier, Stacking, AdaBoost, RealAdaBoost, LPBoost, TotalBoost, BrownBoost, MadaBoost, LogitBoost, GentleBoost, RobustBoost, bucket of models, ensemble learning algorithms, fuzzy logic, Support Vector Machine (SVM), kernelized SVM, Linear classifier, Quadratic Discriminant Analysis (QDA) classifier, Naive Bayes Classifier and Generalized Likelihood Ratio Test (GLRT) classifier with plug-in parametric or non-parametric class conditional density estimation, k-nearest neighbor, Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers, or the like, and any combination thereof. Each possibility is a separate embodiment.

According to some embodiments, the non-linear regression combination of nociception-related physiological parameters may be made using a random-forest algorithm. According to some embodiments, the non-linear regression combination of nociception-related physiological parameters may be made using a boosting framework and/or ensemble learning algorithms.

According to some embodiments, the term “NS value” may refer to a nociception value obtained for a specific patient, having reference to the NS index. According to some embodiments, the NS value is essentially unaffected by physiological changes resulting from administration of an anesthetic and/or analgesic and not from nociception. That is, administration of an anesthetic and/or administration of analgesic, in the absence of pain, does not cause significant changes in the computed NS value. As used herein, the term “essentially” with regards to an unaffected NS value may refer to an NS value which does not qualitatively change the assessment of the patient's nociception level. It is thus understood that the NS value is reflective of nociception per se and is not affected by changes in physiological parameters resulting from drug administration According to some embodiments, an essentially unchanged NS value may include small numerical changes in the NS value as a result of drug administration. According to some embodiments, an essentially unchanged NS value may include a less than 10 unit, less than a 5 unit, or less than a 2 unit change in the NS value as a result of drug administration, out of a 0-100 numerical scale. Each possibility is a separate embodiment.

According to some embodiments, the computing unit may be configured to filter and/or level out side effects and/or physiological changes (e.g. bradycardia and/or vasodilation) caused by administration of a medicament. According to some embodiments, computing the NS value may include leveling out physiological changes caused by drugs. As used herein, the term “level out” may refer to computing the NS value while taking into account a sufficient amount of parameters and or features derived therefrom in order for the NS value to be essentially unaffected by the physiological changes. According to some embodiments, computing the NS value may include filtering out physiological changes caused by medicaments independently of nociception. According to some embodiments, computing the NS value may include providing lower weights to parameters known to change as a result of drug administration. As a non-limiting example, heart rate related parameters and/or features derived therefrom may receive lower weights when the patient is administered remifentanil known to cause bradycardia. As another non-limiting example, photoplethysmogram related parameters and/or features derived therefrom may receive lower weights when the patient is administered propofol known to have vasodilating effects.

According to some embodiments, when the NS value crosses a predefined threshold value nociception is identified. According to some embodiments, when the NS value crosses a first predefined threshold value a mild nociception level is identified. According to some embodiments, when the NS value crosses a second predefined threshold value a moderate nociception level is identified. According to some embodiments, when the NS value crosses a third predefined threshold value a severe nociception level is identified.

According to some embodiments, the NS value enables differentiation between no nociception and nociception with a sensitivity of above 70% at a specificity of at least 70%. According to some embodiments, the NS value enables differentiation between no nociception, mild nociception, moderate nociception and/or severe nociception with a sensitivity of above 70% at a specificity of at least 70%. According to some embodiments, the NS value enables differentiation between no nociception, and severe nociception with a sensitivity of above 70% at a specificity of at least 70%. According to some embodiments, the NS value enables differentiation between no nociception and moderate nociception with a sensitivity of above 70% at a specificity of at least 70%. According to some embodiments, the NS value enables differentiation between no nociception and mild nociception with a sensitivity of above 70% at a specificity of at least 70%. According to some embodiments, the NS value enables differentiation between mild nociception and severe nociception with a sensitivity of above 70% at a specificity of at least 70%. According to some embodiments, the NS value enables differentiation between mild nociception and moderate nociception with a sensitivity of above 70% at a specificity of at least 70%. According to some embodiments, the NS value enables differentiation between moderate nociception and severe nociception with a sensitivity of above 70% at a specificity of at least 70%.

According to some embodiments, the NS value enables differentiation between no nociception and nociception with a sensitivity of above 70% at a specificity of at least 75%. According to some embodiments, the NS value enables differentiation between no nociception, mild nociception, moderate nociception and/or severe nociception with a sensitivity of above 70% at a specificity of at least 75%. According to some embodiments, the NS value enables differentiation between no nociception, and severe nociception with a sensitivity of above 70% at a specificity of at least 75%. According to some embodiments, the NS value enables differentiation between no nociception and moderate nociception with a sensitivity of above 70% at a specificity of at least 75%. According to some embodiments, the NS value enables differentiation between no nociception and mild nociception with a sensitivity of above 70% at a specificity of at least 75%. According to some embodiments, the NS value enables differentiation between mild nociception and severe nociception with a sensitivity of above 70% at a specificity of at least 75%. According to some embodiments, the NS value enables differentiation between mild nociception and moderate nociception with a sensitivity of above 70% at a specificity of at least 75%. According to some embodiments, the NS value enables differentiation between moderate nociception and severe nociception with a sensitivity of above 70% at a specificity of at least 75%.

According to some embodiments, the NS value enables differentiation between no nociception and nociception with a sensitivity of above 75% at a specificity of at least 75%. According to some embodiments, the NS value enables differentiation between no nociception, mild nociception, moderate nociception and/or severe nociception with a sensitivity of above 75% at a specificity of at least 75%. According to some embodiments, the NS value enables differentiation between no nociception, and severe nociception with a sensitivity of above 75% at a specificity of at least 75%. According to some embodiments, the NS value enables differentiation between no nociception and moderate nociception with a sensitivity of above 75% at a specificity of at least 75%. According to some embodiments, the NS value enables differentiation between no nociception and mild nociception with a sensitivity of above 75% at a specificity of at least 75%. According to some embodiments, the NS value enables differentiation between mild nociception and severe nociception with a sensitivity of above 75% at a specificity of at least 75%. According to some embodiments, the NS value enables differentiation between mild nociception and moderate nociception with a sensitivity of above 75% at a specificity of at least 75%. According to some embodiments, the NS value enables differentiation between moderate nociception and severe nociception with a sensitivity of above 75% at a specificity of at least 75%.

According to some embodiments, the NS value enables differentiation between no nociception and nociception with a sensitivity of above 80% at a specificity of at least 75%. According to some embodiments, the NS value enables differentiation between no nociception, mild nociception, moderate nociception and/or severe nociception with a sensitivity of above 80% at a specificity of at least 75%. According to some embodiments, the NS value enables differentiation between no nociception, and severe nociception with a sensitivity of above 80% at a specificity of at least 75%. According to some embodiments, the NS value enables differentiation between no nociception and moderate nociception with a sensitivity of above 80% at a specificity of at least 75%. According to some embodiments, the NS value enables differentiation between no nociception and mild nociception with a sensitivity of above 80% at a specificity of at least 75%. According to some embodiments, the NS value enables differentiation between mild nociception and severe nociception with a sensitivity of above 80% at a specificity of at least 75%. According to some embodiments, the NS value enables differentiation between mild nociception and moderate nociception with a sensitivity of above 80% at a specificity of at least 75%. According to some embodiments, the NS value enables differentiation between moderate nociception and severe nociception with a sensitivity of above 80% at a specificity of at least 75%.

According to some embodiments, the NS value enables differentiation between no nociception and nociception with a sensitivity of above 85% at a specificity of at least 75%. According to some embodiments, the NS value enables differentiation between no nociception, mild nociception, moderate nociception and/or severe nociception with a sensitivity of above 85% at a specificity of at least 75%. According to some embodiments, the NS value enables differentiation between no nociception, and severe nociception with a sensitivity of above 85% at a specificity of at least 75%. According to some embodiments, the NS value enables differentiation between no nociception and moderate nociception with a sensitivity of above 85% at a specificity of at least 75%. According to some embodiments, the NS value enables differentiation between no nociception and mild nociception with a sensitivity of above 85% at a specificity of at least 75%. According to some embodiments, the NS value enables differentiation between mild nociception and severe nociception with a sensitivity of above 85% at a specificity of at least 75%. According to some embodiments, the NS value enables differentiation between mild nociception and moderate nociception with a sensitivity of above 85% at a specificity of at least 75%. According to some embodiments, the NS value enables differentiation between moderate nociception and severe nociception with a sensitivity of above 85% at a specificity of at least 75%.

According to some embodiments, the NS value enables differentiation between no nociception and nociception with a sensitivity of above 85% at a specificity of at least 75%. According to some embodiments, the NS value enables differentiation between no nociception, mild nociception, moderate nociception and/or severe nociception with a sensitivity of above 85% at a specificity of at least 75%. According to some embodiments, the NS value enables differentiation between no nociception, and severe nociception with a sensitivity of above 85% at a specificity of at least 75%. According to some embodiments, the NS value enables differentiation between no nociception and moderate nociception with a sensitivity of above 85% at a specificity of at least 75%. According to some embodiments, the NS value enables differentiation between no nociception and mild nociception with a sensitivity of above 85% at a specificity of at least 75%. According to some embodiments, the NS value enables differentiation between mild nociception and severe nociception with a sensitivity of above 85% at a specificity of at least 75%. According to some embodiments, the NS value enables differentiation between mild nociception and moderate nociception with a sensitivity of above 85% at a specificity of at least 75%. According to some embodiments, the NS value enables differentiation between moderate nociception and severe nociception with a sensitivity of above 85% at a specificity of at least 75%. According to some embodiments, the NS value enables differentiation between no nociception and severe nociception with a sensitivity of above 80% at a specificity of at least 84%. According to some embodiments, the NS value enables differentiation between mild nociception and severe nociception with a sensitivity of above 80% at a specificity of at least 84%. According to some embodiments, the NS value enables differentiation between no nociception, and nociception with a sensitivity of above 80% at a specificity of at least 84%. According to some embodiments, the NS value enables differentiation between no nociception and mild nociception with a sensitivity of above 80% at a specificity of at least 84%. According to some embodiments, the NS value enables differentiation between no nociception and severe nociception with a sensitivity of above 80% at a specificity of at least 84%. According to some embodiments, the NS value enables differentiation between mild nociception and severe nociception with a sensitivity of above 80% at a specificity of at least 84%.

According to some embodiments, the NS value enables differentiation between no nociception and severe nociception with a sensitivity of above 85% at a specificity of at least 84%. According to some embodiments, the NS value enables differentiation between mild nociception and severe nociception with a sensitivity of above 85% at a specificity of at least 84%. According to some embodiments, the NS value enables differentiation between no nociception, and nociception with a sensitivity of above 85% at a specificity of at least 84%. According to some embodiments, the NS value enables differentiation between no nociception and mild nociception with a sensitivity of above 85% at a specificity of at least 84%. According to some embodiments, the NS value enables differentiation between no nociception and severe nociception with a sensitivity of above 85% at a specificity of at least 84%. According to some embodiments, the NS value enables differentiation between mild nociception and severe nociception with a sensitivity of above 85% at a specificity of at least 84%.

According to some embodiments, the NS value computed for the patient may enable determining the amount and/or type of an analgesic required to reduce the NS value below a predetermined threshold value. According to some embodiments, the NS value computed for the patient may enable determining the amount and/or type of an analgesic required to maintain nociception levels within a desired target range. According to some embodiments, the NS value may trigger an output signal.

According to some embodiments, the output signal may include triggering an alarm and/or alert. According to some embodiments, the alarm and/or alert may be visual, audible, and/or physical. Each possibility is a separate embodiment. Non-limiting examples of visual alarms/alerts include a flashing light, a displayed message and/or icon and the like. Each possibility is a separate embodiment. According to some embodiments, the output may be visual, audio, tactile, virtual reality or otherwise noticeable. Non-limiting examples of audible alarms/alerts include a sound, a vocal instruction and the like. Each possibility is a separate embodiment. Non-limiting examples of physical alarms/alerts include vibration, shaking and the like. Each possibility is a separate embodiment. According to some embodiments, a different alarm (e.g. different sound, frequency, intensity or type of alarm/alert) may be triggered when the NS value crosses a first threshold value indicative of a first level of nociception (e.g. mild nociception) as compared to when the NS value crosses a second threshold value indicative of a second level of nociception (e.g. severe nociception). As a non-limiting example, when the NS value crosses the first threshold value a written message may be displayed whereas when the NS value crosses the second threshold value an audial alarm may be triggered. As another non-limiting example, when the NS value crosses the first threshold value an audial alarm at a first intensity may be triggered whereas when the NS value crosses the second threshold value the audial alarm may be triggered at a second intensity (e.g. louder).

According to some embodiments, the output signal may include displaying a written recommendation, for example, a recommendation suggesting that a change in nociception/analgesia management is required. According to some embodiments, the display may be visual, audio, tactile, virtual reality or otherwise noticeable. As a non-limiting example, an NS value representing mild nociception may trigger an output signal suggesting that a moderate increase in analgesic is needed, whereas an NS value indicating that the patient is suffering from severe pain may trigger an output signal indicating that a significant change in analgesic (type and/or amount) is needed. According to some embodiments, the output signal may trigger automatic adjustment of an analgesic dose. For example, an NS value below (or above—depending on the scale) a predetermined threshold may automatically trigger a reduction in the dose of the analgesic administered, whereas an NS value above (or below—depending on the scale) may automatically trigger an increase in the dose of the analgesic administered.

According to some embodiments, there is provided a method for determining a nociception level of a subject, the method including receiving at least three monitored physiological parameters of the patient and computing a nociception scale (NS) value based on an analysis of the at least three parameters and/or features derived therefrom. According to some embodiments, the analysis may include an integrative analysis of the at least three parameters and/or features derived therefrom. According to some embodiments, the method may include leveling out changes in physiological parameters caused by administration of drugs rather than nociception. As a non-limiting example, the method may include, directly or indirectly, leveling out changes in heart rate related parameters caused due to a drug's (e.g. remifentanil) bradycardial effect. According to some embodiments, when the NS value crosses a first predefined threshold value a mild nociception level is identified; and when the NS value crosses a second predefined threshold value a severe nociception level is identified. According to some embodiments, the method further includes triggering an alarm when the NS value crosses the first and/or second predefined threshold value, as essentially described herein.

According to some embodiments, there is provided a computer implemented software configured to determine a nociception level of a subject. According to some embodiments, the software may be configured to receive at least three monitored physiological parameters from at least one functionally connected sensor and to compute a nociception scale (NS) value based on an analysis of the at least three parameters and/or features derived therefrom. According to some embodiments, the analysis may include an integrative analysis of the at least three parameters and/or features derived therefrom. According to some embodiments, the software may be further configured to level out changes in physiological parameters caused by administration of drugs in a manner independent of nociception. As a non-limiting example, the software may be configured to, directly or indirectly, level out changes in plethysmography (PPG) related parameters caused due to a drug's (e.g. remifentanil) bradycardial effect and not due to changes in nociception. According to some embodiments, the software may be configured to trigger an alarm when the NS value crosses the first and/or second predefined threshold value, as essentially described herein.

According to some embodiments, there is provided a device for determining analgesic efficacy. According to some embodiments, the device includes at least one sensors configured to sense at least three parameters of a patient. According to some embodiments, the at least three parameters may be sensed before (A0) and after (A1) administration of an analgesic and before (S0) and after (S1) providing a noxious stimulus. It is understood to one of ordinary skill in the art that some measurements may represent combinations of A0, A1, S0 or S1. For example, A0 and S0 may be obtained from a measurement made before administration of the analgesic and before the noxious stimulus has been provided. Similarly, A0 and S1 may be obtained from a measurement made before administering a drug but after providing the noxious stimulus. For example, A1 and S0 may be obtained from a measurement made after administration of the analgesic but before the noxious stimulus; and A1 and S1 may be obtained from a measurement made after administration of the analgesic and after the noxious stimulus. It is further understood that the at least three parameters may be sensed before and after administering additional doses of analgesics (A2, A3 . . . An) and/or before and after providing additional noxious stimuli (S2, S3 . . . Sn).

As a non-limiting example, the at least three parameters may be sensed at a first time point t₀, prior to administration of the analgesic and prior to providing a stimulus. At time point t₁, an analgesic may be administered followed by a second measurement of the at least three parameters. It is understood by one of ordinary skill in the art that the time window between administration of the analgesic and the measurement of the at least three parameters may vary depending on the type of analgesic administered and may thus be determined accordingly. At time point t₂, a noxious stimulus may be provided followed by a third measurement of the at least three parameters, typically simultaneously with or immediately after the stimulus.

As another non-limiting example, the at least three parameters may be sensed at a first time point t₀, prior to administration of the analgesic and prior to providing a stimulus. At time point t₁, a noxious stimulus may be provided followed by a second measurement of the at least three parameters, typically simultaneously with or immediately after the stimulus. At time point t₂, an analgesic may be administered followed by a third measurement of the at least three parameters and, at time point t₃, a second noxious stimulus may be provided followed by a third measurement of the at least three parameters, again typically either simultaneously with or immediately after the stimulus.

According to some embodiments, the device further includes a computing unit adapted to receive the at least three parameters from the at least one sensor obtained for A0, A1, S0 and S1 and/or combinations thereof. According to some embodiments, the computing unit is further configured to compute nociception scale (NS) values for A0, A1, S0 and S1 and/or combinations thereof based on an analysis of the at least three parameters and/or features derived therefrom. According to some embodiments, the analysis may include an integrative analysis of the at least three parameters and/or features derived therefrom.

According to some embodiments, the computing unit is further configured to determine the efficacy of the analgesic based on a comparison of the NS values obtained for A0, A1, S0 and S1 and/or combinations thereof. It is understood to one of ordinary skill in the art that in order for an analgesic to be considered efficient, the NS values obtained before administration of the analgesic and before the noxious stimulus (A0S0) should be unchanged or only mildly elevated after administration of the analgesic and after the noxious stimulus (A1S1). According to some embodiments, in order for an analgesic to be considered efficient, the change in the NS value between A0S0 and A1S1 should be less than 10 units out of a 0-100 numerical scale. According to some embodiments, in order for an analgesic to be considered efficient, the change in the NS value between A0S0 and A1S1 should be less than 5 units out of a 0-100 numerical scale. According to some embodiments, in order for an analgesic to be considered efficient, the change in the NS value between A0S0 and A1S1 should be less than 2 units out of a 0-100 numerical scale. According to some embodiments, in order for an analgesic to be considered efficient, the change in the NS value between A0S0 and A1S1 should be less than 1 unit out of a 0-100 numerical scale.

According to some embodiments, the noxious stimulus in absence of an analgesic (A0S1) may cause an at least 10 unit increase in the NS value out of a 0-100 numerical scale, as compared to before the noxious stimulus (A0S0). According to some embodiments, the noxious stimulus in absence of an analgesic (A0S1) may cause an at least 15 unit increase in the NS value out of a 0-100 numerical scale, as compared to before the noxious stimulus (A0S0). According to some embodiments, the noxious stimulus in absence of an analgesic (A0S1) may cause an at least 20 unit increase in the NS value out of a 0-100 numerical scale, as compared to before the noxious stimulus (A0S0). According to some embodiments, the noxious stimulus in absence of an analgesic (A0S0) may cause an at least 30 unit increase in the NS value out of a 0-100 numerical scale, as compared to before the noxious stimulus (A0S0), as compared to before the noxious stimulus (A0S0).

According to some embodiments, administration of the analgesic, in the absence of a noxious stimulus (A1S0), causes essentially no change in the NS value as compared to before the administration of the analgesic (A0S0). According to some embodiments, administration of the analgesic in the absence of a noxious stimulus (A1S0) causes essentially no change in the NS value as compared to before the administration of the analgesic (A0S0), such as less than 10 units, 5 units, 2 units or 1 units change in the NS value out of a 0-100 numerical scale. Each possibility is a separate embodiment.

According to some embodiments, the noxious stimulus may include tetanic stimulus, thermal (heat or cold) stimulus, pressure stimulus, touch (tickle, itch, crude, flutter, pressure) stimulus, electric stimulus, mechanical stimulus, proprioception stimulus, chemical stimulus or combinations thereof. Each possibility is a separate embodiment.

According to additional or alternative embodiments, the device may be configured to determine an efficient dose of an analgesic.

According to some embodiments, when determining an efficient dose of an analgesic, the at least three parameters may be sensed before and after administration of at least one dose of an analgesic and/or before and after providing a noxious stimulus.

According to some embodiments, when determining an efficient dose of an analgesic, the at least three parameters may be sensed before (A0) administration of an analgesic, after a first dose of an analgesic (A1) and after a second dose of an analgesic (A2) before (S0) and after (S1) providing a noxious stimulus. It is understood to one of ordinary skill in the art that some measurements may represent combinations of A0, A1, A2, S1 or S2, as essentially described above. It is further understood that any suitable number of doses may be tested and that the first and second doses are illustrative only.

According to some embodiments, in order for a dose to be considered efficient, the change in the NS value between A0S0and A1S1 or A2S1 should be less than 10 units, less than 5 units, less than 2 units or less than 1 units. Each possibility is a separate embodiment.

According to some embodiments, in order for a dose of an analgesic to be considered efficient, the NS value before and after a noxious stimuli should be essentially unchanged (i.e. enough to avoid nociception caused by the stimuli), while the NS value of a lower dose causes changes in the NS value, i.e. the dose is the lowest possible dose required to avoid nociception. According to some embodiments, in order for a dose to be considered efficient, the change in the NS value between A0S0 and A2S1 should be less than 10 units, less than 5 units, less than 2 units or less than 1 units, whereas the change in the NS value between A0S0 and A1S1 is larger than 1 unit, 2 units, 5 units 10 units, 20 units or 30 units. Each possibility and/or combination are separate embodiments.

According to some embodiments, there is provided a method for determining efficacy of an analgesic, the method including receiving at least three monitored physiological parameters before (A0) and after (A1) administration of an analgesic and before (S0) and after (S1) providing a noxious stimulus, computing nociception scale (NS) values for A0, A1, S0 and S1 and/or combinations thereof based on an analysis of the at least three parameters and/or features derived therefrom and determining the efficacy of the analgesic based on a comparison of the NS values obtained for A0, A1, S0 and S1 and/or combinations thereof, as essentially described herein. According to some embodiments, the analysis may include an integrative analysis.

According to some embodiments, there is provided a computer implemented software configured to determine efficacy of an analgesic. According to some embodiments, the software may be configured to receive at least three physiological parameters monitored before (A0) and after (A1) administration of an analgesic and before (S0) and after (S1) providing a noxious stimulus to compute a nociception scale (NS) value for A0, A1, S0 and S1 and/or combinations thereof and to determine the efficacy of the analgesic based on a comparison of the NS values obtained for A0, A1, S0 and S1 and/or combinations thereof, as essentially described herein.

According to some embodiments, there is provided a method for determining an optimal dose of an analgesic, the method including receiving at least three monitored physiological parameters before (A0) administration of an analgesic and after administration of a first (A1) and a second (A2) dose of an analgesic and before and after (S0) and after (S1) providing a noxious stimulus, computing nociception scale (NS) values for A0, A1, A2, S0 and S1 and/or combinations thereof based on an analysis of the at least three parameters and/or features derived therefrom and determining the optimal dose of the analgesic based on a comparison of the NS values obtained for A0, A1, A2, S0 and S1 and/or combinations thereof, as essentially described herein. According to some embodiments, the analysis may include an integrative analysis.

According to some embodiments, there is provided a computer implemented software configured to determine an optimal dose of an analgesic. According to some embodiments, the software may be configured to receive at least three physiological parameters monitored before (A0) administration of an analgesic and after administration of at least a first (A1) and a second (A2) dose of an analgesic and before (S0) and after (S1) providing at least one noxious stimulus, to compute a nociception scale (NS) value for A0, A1, A2, S0 and S1 and/or combinations thereof and to determine the optimal dose of the analgesic based on a comparison of the NS values obtained for A0, A1, A2, S0 and S1 and/or combinations thereof, as essentially described herein.

According to some embodiments, there is provided a computer implemented software configured to determine an optimal dose of an analgesic. According to some embodiments, the software may be configured to receive at least three physiological parameters monitored before and after administration of at least one dose of an analgesic and/or before and after providing at least one noxious stimulus, to compute corresponding nociception scale (NS) values and to determine the optimal dose of the analgesic based on the NS values obtained, as essentially described herein.

According to some embodiments, determining an optimal dose of an analgesic includes determining a nociception profile of the patient. According to some embodiments, the optimal dose of an analgesic may be customized to the specific patient.

According to some embodiments, there is provided a method for determining a nociception profile of a patient. According to some embodiments, the method may include computing a baseline nociception scale (NS) value of the patient, providing a first noxious stimuli to a patient, computing a first nociception scale (NS) value in response to the first noxious stimuli, providing a first dose of an analgesic to the patient, providing a second noxious stimuli to a patient, and computing a second nociception scale (NS) value in response to the second noxious stimuli. According to some embodiments, the first and second noxious stimuli may be identical. According to some embodiments, the first and second noxious stimuli may be different (e.g. different intensity and/or different type of stimuli). It is understood by one of ordinary skill in the art, that the method may include providing additional stimuli and/or additional doses of analgesic, thereby enhancing the resolution of the patient's nociception profile.

According to some embodiments, the operation of the stimulator (e.g. an electrical, thermal, mechanical, or other stimulator) may be automated and controlled by an internal or external processing unit. According to some embodiments, adjustments in the noxious stimulus provided (e.g. changes in the type of stimulus, and or changes in the length and/or frequency of a stimulus) may be automated. For example, the operation and/or changes in operation of the stimulator may be based on input signals automatically received by the processing unit. For example, the operation and or changes in operation of the stimulator may be based on a predefined operation program encoded into the stimulator. Additionally or alternatively, the dosages of analgesics may be automatically provided and/or adjusted based on control signals sent to an infusion pump providing the analgesic dosages to the patient. Before explaining at least one embodiment in detail, it is to be understood that aspects of the embodiments are not necessarily limited in their application to the details of construction and the arrangement of the components and/or methods set forth herein. Some embodiments may be practiced or carried out in various ways. The phraseology and terminology employed herein are for descriptive purposes and should not be regarded as limiting.

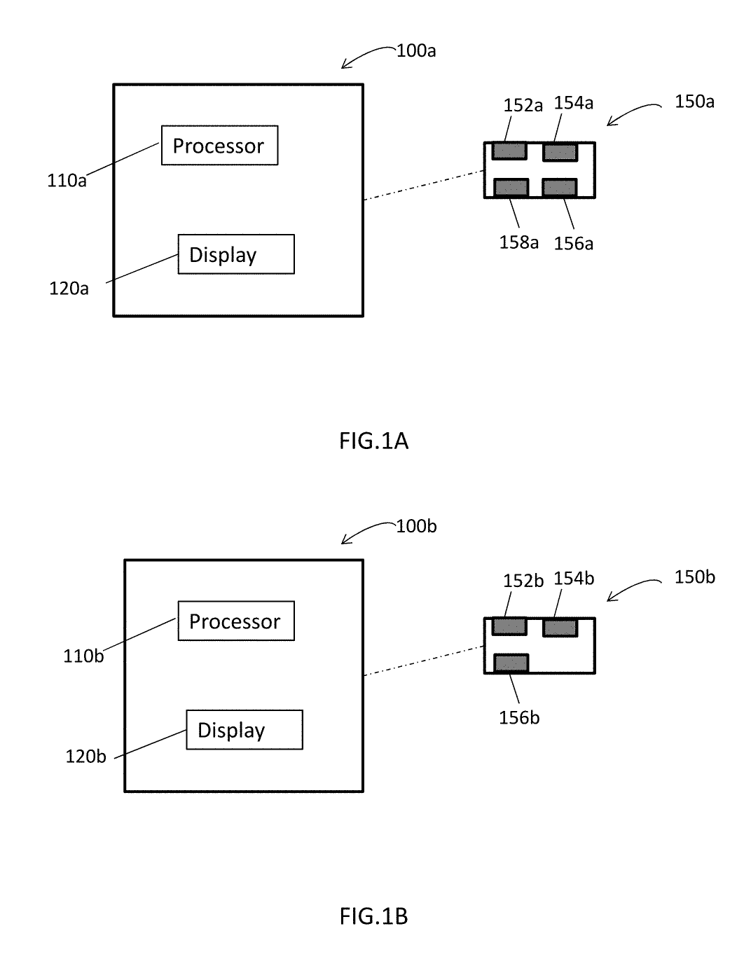

Reference is now made to FIG. 1A, which schematically illustrates a nociception/analgesia monitoring medical device 100a, according to some embodiments. Medical device 100a is functionally connected (wired or wirelessly (Bluetooth, zigbi, wifi, etc.) to a finger probe 150a, which includes physiological sensors, here PPG sensor 152a, accelerometer 154a, GSR sensor 156a and skin temperature sensor 158a. Medical device 100a includes a processor 110a configured to receive physiological signals and/or parameters from PPG sensor 152a, accelerometer 154a, GSR sensor 156a and skin temperature sensor 158a and to compute a nociception scale (NS) value based on an analysis of the received signals and/or parameters. According to some embodiments, processor 110a is configured to extract features from the received physiological signals and/or parameters based upon which the nociception scale (NS) value is computed. According to some embodiments, processor 110a filters and/or levels out changes in physiological parameters caused by side effects of drugs administered to the patient, such as vasodilation and bradycardia. Medical device 100a may further include a display 120a configured to display the NS value and/or changes therein. According to some embodiments, finger probe 150a, PPG sensor 152a, accelerometer 154a, GSR sensor 156a and/or skin temperature sensor 158a and/or processor 110a may include an analog to a digital (A2D) converter (not shown) configured to transfer the obtained physiological signals and/or parameters into digital signals. It is understood that if the A2D converter is incorporated into finger probe 150a and/or into PPG sensor 152a, accelerometer 154a, GSR sensor 156a and/or skin temperature sensor 158a, processor 110a may be a virtual processor, such as an internet enabled device (i.e. cloud computing) as well as an external processor and/or a processor located on/within finger probe 150a.

Reference is now made to FIG. 1B, which schematically illustrates a nociception/analgesia monitoring medical device 100b, according to some embodiments. Medical device 100b is functionally connected (wired or wirelessly (Bluetooth, zigbi, wifi, etc.) to a sensor unit 150b, such as a wristband, which includes physiological sensors, here respiration sensor 152b, electrocardiograph (EEG) 154b and blood pressure sensor 156b. Medical device 100b includes a processor 110b configured to receive physiological signals and/or parameters from respiration sensor 152b, electrocardiograph (EEG) 154b and blood pressure sensor 156b an and to compute a nociception scale (NS) value based on an analysis of the received signals and/or parameters. According to some embodiments, processor 110b is configured to extract features from the received physiological signals and/or parameters based upon which the nociception scale (NS) value is computed. According to some embodiments, processor 110b filters and/or levels out changes in physiological parameters caused by side effects of drugs administered to the patient, such as vasodilation and bradycardia. Medical device 100b may further include a display 120b configured to display the NS value and/or changes therein. According to some embodiments, sensor unit 150b, respiration sensor 152b, electrocardiograph (EEG) 154b, blood pressure sensor 156b and/or processor 110b may include an analog to a digital (A2D) converter (not shown) configured to transfer the obtained physiological signals and/or parameters into digital signals. It is understood that if the A2D converter is incorporated into sensor unit 150b and/or into respiration sensor 152b, electrocardiograph (EEG) 154b and/or blood pressure sensor 156b, processor 110b may be a virtual processor, such as an internet enabled device (i.e. cloud computing) as well as an external processor and/or a processor located on/within sensor unit 150b.

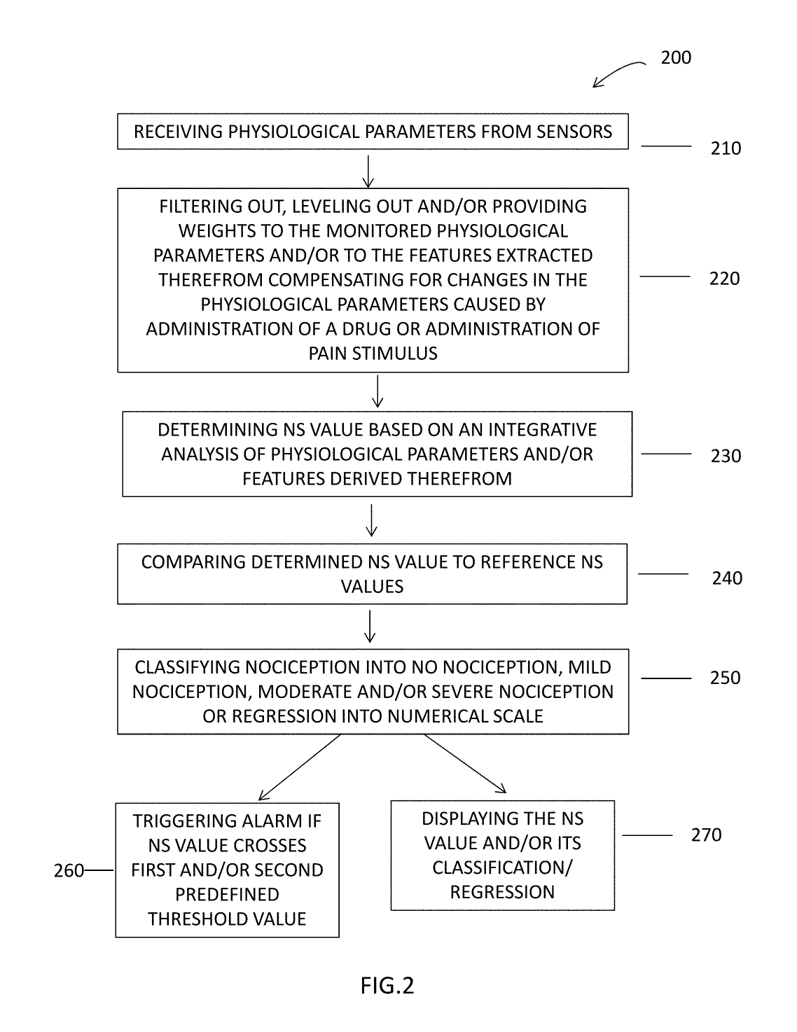

Reference is now made to FIG. 2, which is an illustrative flowchart of a method 200 for determining a nociception scale (NS) value, according to some embodiments. In step 210 physiological parameters are received from physiological sensors, such as, but not limited to a PPG sensor, GSR sensor, skin temperature sensor and/or an accelerometer or other parameters or combination of parameters. In step 220 the method may optionally include filtering out, leveling out and/or providing weights to the monitored physiological parameters and/or to the features extracted therefrom compensating for changes in the parameters caused by administration of a drug, as essentially described herein. It is understood that the filtering out, leveling out and/or providing weights to the monitored physiological parameters may be performed prior to, after or simultaneously with the determination of the NS value. It is further understood that the filtering out, leveling out and/or providing weights to the monitored physiological parameters may be an integral part of computing the NS value and/or may be a separate step performed independently of the computation of the NS value. In step 230, an NS value is determined based on an analysis of the at least three physiological parameters and/or features derived therefrom. According to some embodiments, method 200 further includes an additional step 240 in which the determined NS value is compared to reference NS values and optionally step 250 in which classification into no nociception, mild nociception, moderate nociception and/or severe nociception is implemented and/or regression into a scale, such as but not limited to a scale of 0-100 or 0 . . . 10, etc., as essentially described herein. Optionally, in step 260 an alarm/alert is triggered when the NS value crosses the first and/or second predefined threshold value, as essentially described herein. Additionally or alternatively, optional step 270 may display the NS value, its classification and/or its regression on a display.

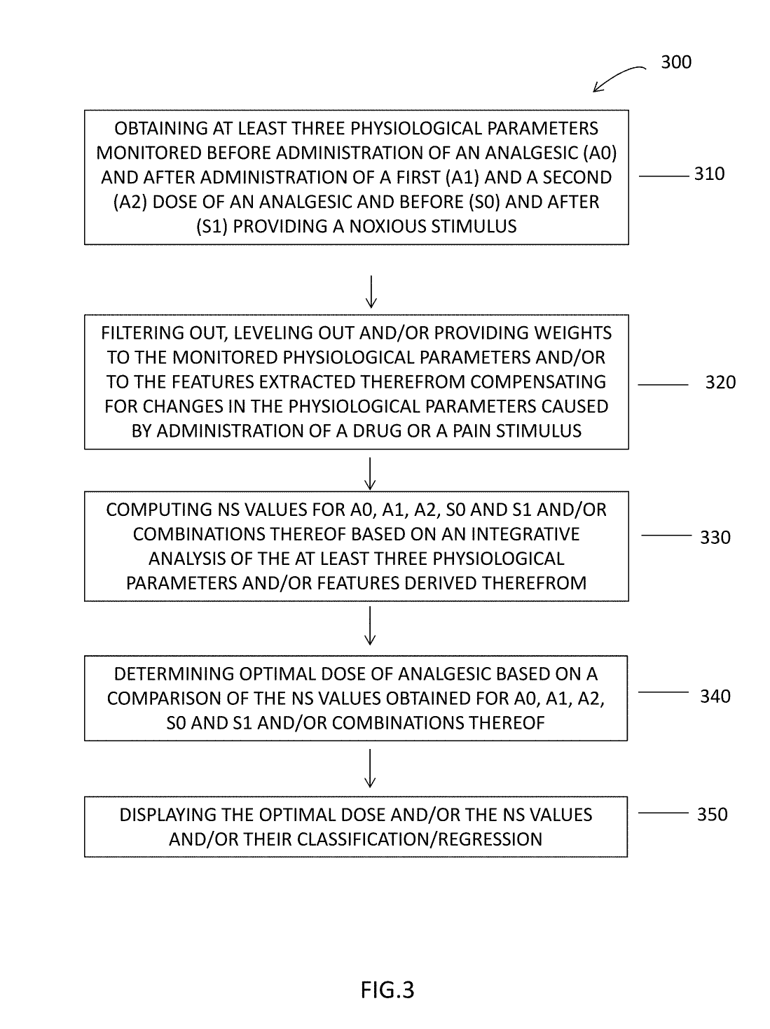

Reference is now made to FIG. 3, which is an illustrative flowchart of a method 300 for determining an optimal dose of an analgesic, according to some embodiments. In step 310 at least three physiological parameters monitored before administration of an analgesic (A0) and after administration of a first (A1) and a second (A2) dose of an analgesic as well as before and after (S0) and after (S1) providing a noxious stimulus, as essentially described herein. In step 320, the method may optionally include filtering out, leveling out and/or providing weights to the monitored physiological parameters and/or to the features extracted therefrom, compensating for changes in the parameters caused by administration of a drug, as essentially described herein. It is understood that the filtering out, leveling out and/or providing weights to the monitored physiological parameters may be performed prior to, after or simultaneously with the determination of the NS value. It is further understood that the filtering out, leveling out and/or providing weights to the monitored physiological parameters may be an integral part of computing the NS value and/or may be a separate step performed independently of the computation of the NS value. In step 330, NS values for A0, A1, A2, S0 and S1 and/or combinations thereof are computed based on an analysis of the at least three physiological parameters and/or features derived therefrom, as essentially described herein. In step 340 an optimal dose of the analgesic is determined based on a comparison of the NS values obtained for A0, A1, A2, S0 and S1 and/or combinations thereof, as essentially described herein. It is understood that in order for a dose of an analgesic to be considered efficient, the NS value before and after a noxious stimuli should be essentially unchanged (i.e. enough to avoid nociception caused by the stimuli), while the NS value of a lower dose causes changes in the NS value, i.e. the dose is the lowest possible dose required to avoid nociception, as essentially described herein. Optionally, step 350 may include displaying the effective dose, the NS value, its classification and/or its regression on a display.

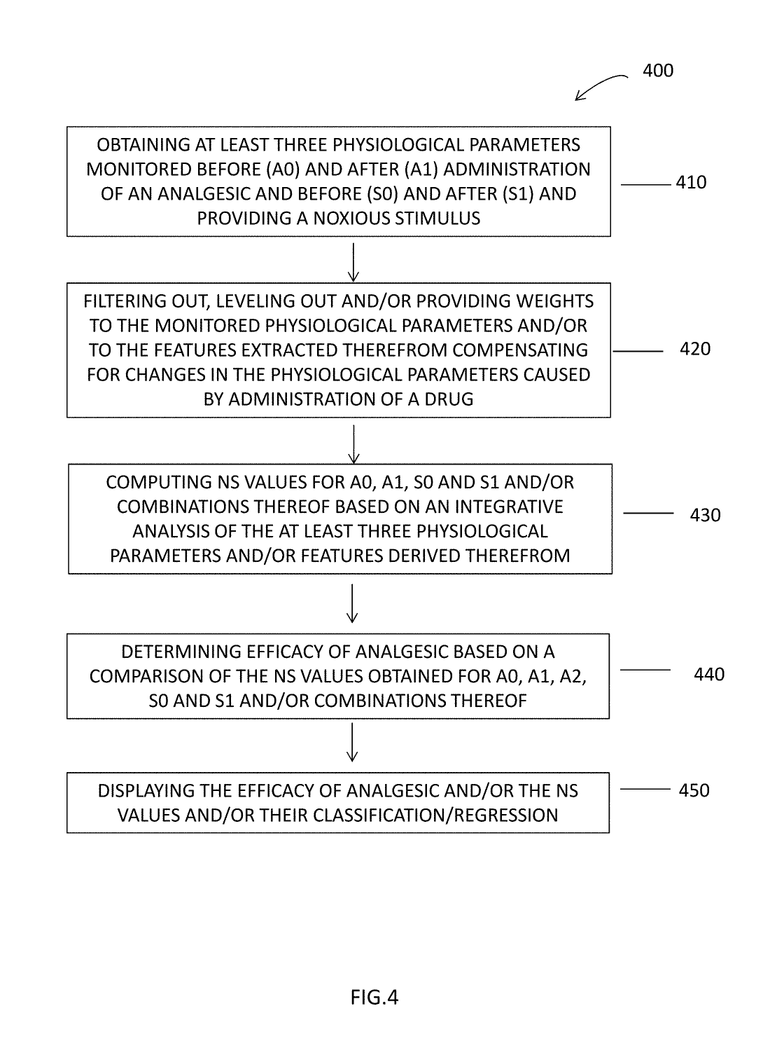

Reference is now made to FIG. 4, which is an illustrative flowchart of a method 400 for determining efficacy of an analgesic, according to some embodiments. In step 410 at least three physiological parameters monitored before (A0) and after (A1) administration of an analgesic as well as before and after (S0) and after (S1) providing a noxious stimulus, as essentially described herein. In step 420 the method may optionally include filtering out, leveling out and/or providing weights to the monitored physiological parameters and/or to the features extracted therefrom, compensating for changes in the parameters caused by administration of a drug, as essentially described herein. It is understood that the filtering out, leveling out and/or providing weights to the monitored physiological parameters may be performed prior to, after or simultaneously with the determination of the NS value. It is further understood that the filtering out, leveling out and/or providing weights to the monitored physiological parameters may be an integral part of computing the NS value and/or may be a separate step performed independently of the computation of the NS value. In step 430, NS values for A0, A1, S0 and S1 and/or combinations thereof are computed based on an analysis of the at least three parameters and/or features derived therefrom, as essentially described herein. In step 440 efficacy of the analgesic is determined based on a comparison of the NS values obtained for A0, A1, S0 and S1 and/or combinations thereof, as essentially described herein. It is understood to one of ordinary skill in the art that in order for an analgesic to be considered efficient the NS values obtained before administration of the analgesic and before the noxious stimulus (A0S0) should be unchanged or only mildly elevated after administration of the analgesic and after the noxious stimulus (A1S1), as essentially described herein. Optionally, step 450 may include displaying the efficacy of the analgesic, the NS value, its classification and/or its regression on a display.

Reference is now made to FIG. 5, which is an illustrative flowchart of a method 500 for determining the amount of an analgesic required to maintain nociception levels within a desired target range. In step 510 at least three physiological parameters monitored for a patient undergoing a pain inflicting medical intervention. In step 520 the method may optionally include filtering out, leveling out and/or providing weights to the monitored physiological parameters and/or to the features extracted therefrom, compensating for changes in the parameters caused by administration of a drug, as essentially described herein. It is understood that the filtering out, leveling out and/or providing weights to the monitored physiological parameters may be performed prior to, after or simultaneously with the determination of the NS value. It is further understood that the filtering out, leveling out and/or providing weights to the monitored physiological parameters may be an integral part of computing the NS value and/or may be a separate step performed independently of the computation of the NS value. In step 530, an NS value is determined based on an analysis of the at least three parameters and/or features derived therefrom. In step 540, the amount of an analgesic required to maintain nociception levels within a desired target range is determined. According to some embodiments, determining the amount of analgesic required to reduce the NS value below a predetermined threshold value may include comparing to a library of pre-stored data obtained for the specific analgesic and/or for the deviance of the NS value from the predetermined threshold value. Optionally, step 550 may include displaying the required amount of the analgesic, the NS value, its classification and/or its regression on a display.

The following examples are presented in order to more fully illustrate some embodiments of the invention. They should, in no way be construed, however, as limiting the broad scope of the invention.

### EXAMPLES

### Example 1

### Clinical Study #1

### Ethics

The protocol was performed after approval was obtained from the local Human Ethics Committee (Commissie Medische Ethiek, Leiden University Medical Center, 2300 RC Leiden, The Netherlands) and was registered at www.clinicaltrials.gov under number NCT01912118. All patients gave oral and written informed consent before enrollment into the study. The study was performed from July 2013 to June 2014.

### Patients

American Society of Anesthesiology class I, II or III patients (aged 18-80 years) of either sex, scheduled for elective surgery under general anesthesia, were recruited to participate in the study. Exclusion criteria included: inability to give informed consent, pregnancy or lactation, body mass index >35 kg/m2, perceived difficult intubation, planned rapid sequence intubation and use of beta-adrenergic receptor antagonists. Preoperative preparation was according to local protocol.

### Study Design

In this prospective randomized study, patients received total intravenous anesthesia with propofol and remifentanil. Seventy-two patients were randomly assigned to one of six possible remifentanil target concentrations: 0 (propofol only, n=12), 1, (n=12), 2 (n=12), 3 (n=12), 4 (n=12) and 5 (n =12) ng/mL using a custom built remifentanil target controlled infusion pump (Remifusor, University of Glasgow, United Kingdom) programmed with the remifentanil pharmacokinetic data set published by Minto et al. Similarly, propofol was infused using a target controlled infusion system (Orchestra Base Primea, Fresenius Kabi, Zeist, The Netherlands) programmed with the propofol pharmacokinetic set of Marsh et al. The target was adapted such that prior to intubation or skin incision the bispectral index (BIS) of the electroencephalogram (BIS VISTA, Covidien, Dublin, Ireland) was maintained at 45±5 for at least 10-15 min. If needed a muscle relaxant (rocuronium 0.5 mg/kg) could be administered prior to intubation.

### Data Collection

A finger probe containing a plethysmography (PPG) sensor, a galvanic skin response (GSR) sensor, skin temperature sensor and a 3-axis accelerometer was placed on the index finger of the subject's right hand (Medasense Biometrics, Ramat Yishai, Israel).

The signals from the probe were sampled at 50 Hz and recorded on a laptop and processed off-line using MatlabR2011b software (TheMathworks Inc., Natick, Mass.). The following variables were calculated from the finger probe heart rate (HR), heart rate variability (HRV), plethysmograph wave amplitude (PPGA), skin conductance level (SCL), and fluctuations in skin conductance and their derivatives. To measure the non-invasive beat-to-beat blood pressure, an appropriately sized finger cuff was applied to the mid-phalanx of the left index finger, which was connected to a Nexfin monitor (Edwards Lifesciences, Irvine, Calif.). See Ref. Martina et al. for an elaborate explanation of the Nexfin system and calculation of blood pressure. The beat-to-beat finger blood pressure was stored on disc for off-line analysis. Data were collected from the time of induction of anesthesia until approximately 3-5 minutes after incision. Specific events occurring during the study (start of induction, patient movement, intubation, incision) were logged to enable a direct link between stimulus and measurements.

Description of the Nociception Scale (NS)

The NS is based on a non-linear combination of nociception-related physiological parameters: heart rate (HR), heart rate variability (HRV at the 0.15-0.4 Hz band power), amplitude of the photo-plethysmograph wave (PPGA), skin conductance level (SCL), number of skin conductance fluctuations (NSCF), and their time derivatives. The NS was developed to correlate with a reference clinical score of nociception based on estimated opioid concentration and stimulus. A composite parameter was derived using a non-linear regression method, in which the physiological signals with their derivatives were used as predictor variables and the combined estimated opioid concentration and stimulus were used as the observed variable for the non-linear regression models. The estimated multiparameter composite derived from the regression analyses was scaled from 0 to 100 to produce the NS.

### Data Analysis

The sample size was set to include 12 subjects per remifentanil treatment level i.e. 72 patients in total. Statistical and data analyses were performed using Matlab R2011b scientific software (The Mathworks Inc., Natick, Mass.). Three distinct stimuli were defined in each patient: a non-noxious event, incision (moderate noxious stimuli) and intubation (severe noxious stimuli). A non-noxious event was defined as a 1-min interval within a 5-min window of absence of noxious stimulation; intubation was defined as the time interval around the insertion of the orolaryngeal tube into the trachea and included the preceding laryngoscopy; incision was defined as the time interval around the surgical skin incision. For each stimulus, two parameter values were defined, one prior to stimulation (before) and one after stimulation (after), which were the average of data before and after the stimulus along a certain time interval. These time intervals were for the non-noxious stimulus the first (before) and last (after) 30 s of the 1-min non-noxious interval, for incision and intubation the 30-60 s prior to the stimulus (before) and the 10-180 s following the stimulus (after).

### Statistical Analysis

3 pain assessment indices were compared, namely NS, mean arterial blood pressure (MAP) and heart rate (HR). The following statistical tests were performed to compare the performance of NS, ΔNS, MAP, ΔMAP, HR and ΔHR:

1. Right-tailed paired t-test to assess whether the average reaction (Δ) of the three indices to stimulation are significantly greater than zero. Two-tailed unpaired t-test to assess whether the population values of the variables after stimulation were significantly different from values obtained before stimulation. Additionally the effect of stimulation on BIS was tested using paired and unpaired t-tests.

2. Receiver operating characteristic (ROC) curves were constructed to assess the ability of the individual variables (absolute values and Δ) to discriminate between noxious and non-noxious events. Confidence intervals of the area-under-the-curves (AUC) were calculated using the method of Hanley and McNeil, which corrects for the use of correlated data.

3. Repeated analysis of variance (RM-NOVA) to test the ability of each of the variables to grade noxious stimuli, i.e. to assess whether the variable values increased with an increasing stimulus strength: non-noxious stimulus<moderate noxious stimulus (incision)<intense noxious stimulus (intubation). In case of a significant main effect, a Scheffe's post-hoc multiple comparison test was applied to test between pairs non- noxious stimulus vs. intubation, non-noxious stimulus vs. incision and incision vs. intubation.

4. For non-noxious stimuli and intubation, the Spearman correlation coefficient was calculated to quantify the relation between HR, MAP, NS and the remifentanil target concentration. This was done separately for time intervals before and after. A quadratic polynomial was fitted by least square analysis to the data. Values are presented as mean ±SD or mean (95% confidence interval, CI) unless otherwise indicated; p-values <0.05 were considered significant.

### Results

Seventy-two patients participated in the study, as described. The data from 71 patients were used in the analysis. All patients completed the study without side effects. In about half of the patients, a muscle relaxant was administered before intubation. Prior to noxious stimulation (intubation/skin incision) BIS values were on average 45.0±9.0 (mean±SD), 45.6±9.9, 47.2±9.1, 42.6±7.4, 44.7±8.0 and 47.0±9.8 in the 0, 1, 2, 3, 4 and 5 ng/mL remifentanil groups, respectively (RM-ANOVA: p>0.05; grand mean 45.5±8.8).

### Response to Noxious Events

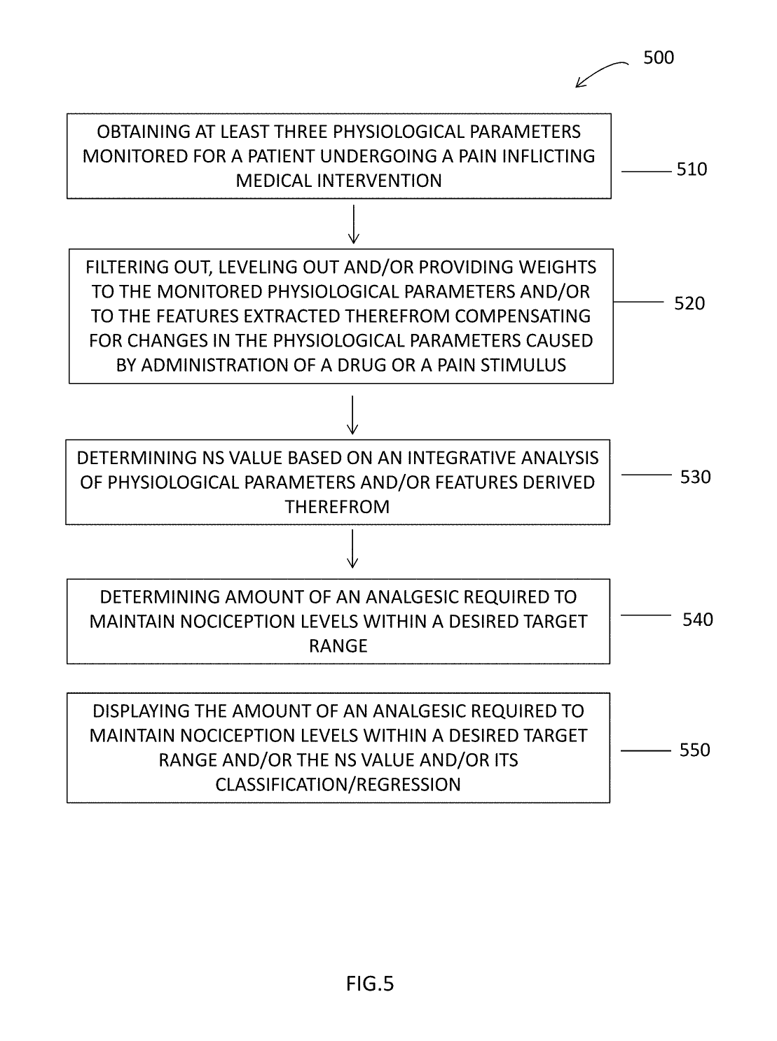

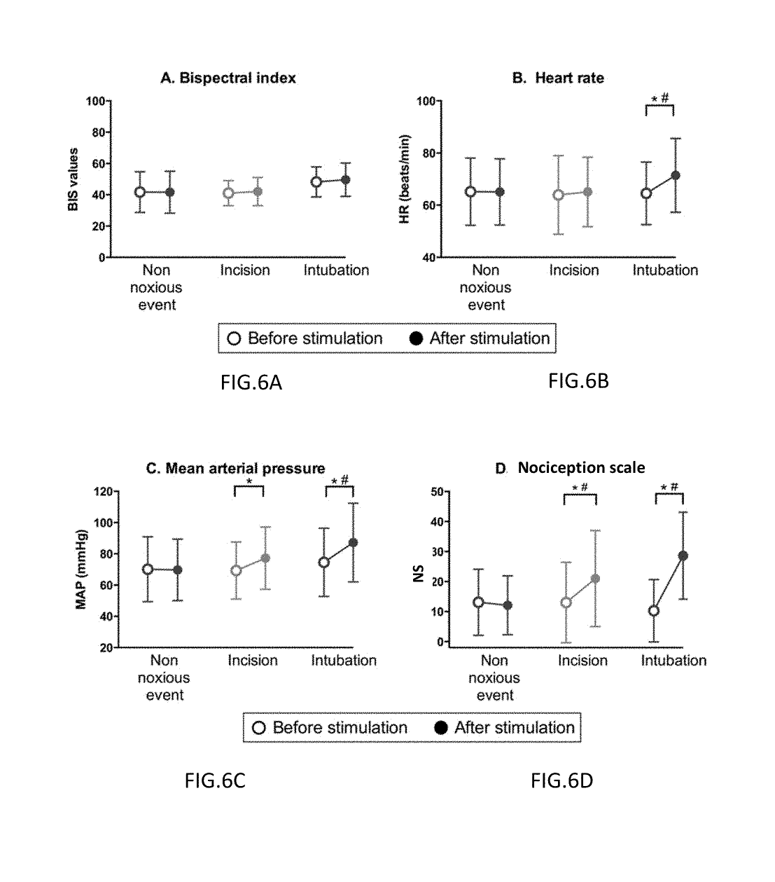

The effect of non-noxious stimuli, incision and intubation on BIS, HR, MAP and NS are given in FIG. 6. Non-noxious stimuli had no effect on any of the variables when comparing before to after time intervals (mean difference (95% CI)): ΔBIS—0.1 (−0.9 to 0.7), ΔHR—0.13 (−0.7 to 0.3) min−1, ΔMAP—0.45 (−1.9 to 2.1) mmHg and ΔNS—1.1 (−3.6 to 2.0). Intubation caused an increase in HR, MAP and NS but not BIS: ΔBIS 1.7 (−3.9 to 6.3; ns), ΔHR 7.0 (1.4 to 12.0; paired t-test p<0.001; unpaired t-test p<0.001) min−1, ΔMAP 13.0 (3.1 to 20; paired t-test: p<0.001; unpaired t-test p<0.001) mmHg and ΔNS 18.0 (7.8 to 29.0; paired t-test: p<0.001; unpaired t-test p<0.001). Paired t-test indicated by * and unpaired t-test indicated by #).

Incision had no effect on BIS and HR but caused increases in MAP and NS although, in contrast to MAP, the effects on NS were significant in both paired and unpaired t-tests: ΔBIS 0.92 (−1.2 to 3.3; ns), ΔHR 1.3 (−0.46 to 3.1; ns) min−1, ΔMAP 7.9 (−1.9 to 13.0; paired t-test: p<0.001; unpaired t-test ns) mmHg and ΔNS 8.0 (0.4 to 16.0; paired t-test: p<0.001; unpaired t-test p<0.001).

Comparing the three different stimuli, a significant pain response observed for HR, MAP and NS after (but not before) stimulation and A's: HR F(2,96)=9.4, p<0.001; ΔHR F(2,96)=27, p<0.0001); MAP F(2,80)=28, p<0.001; ΔMAP F(2,80)=19, p<0.0001; NDI F(2,96)=23, p<0.0001; ΔNS F(2,96)=46, p<0.0001.

Post-hoc analysis showed that only NS (after stimulation) and ΔNS graded the level of noxious intensity with non-noxious NS<incision NS<intubation NS. HR after stimulation and ΔHR could not differentiate between non-noxious stimuli and incision (p=0.24). ΔMAP could not discriminate between incision and intubation (p=0.07).

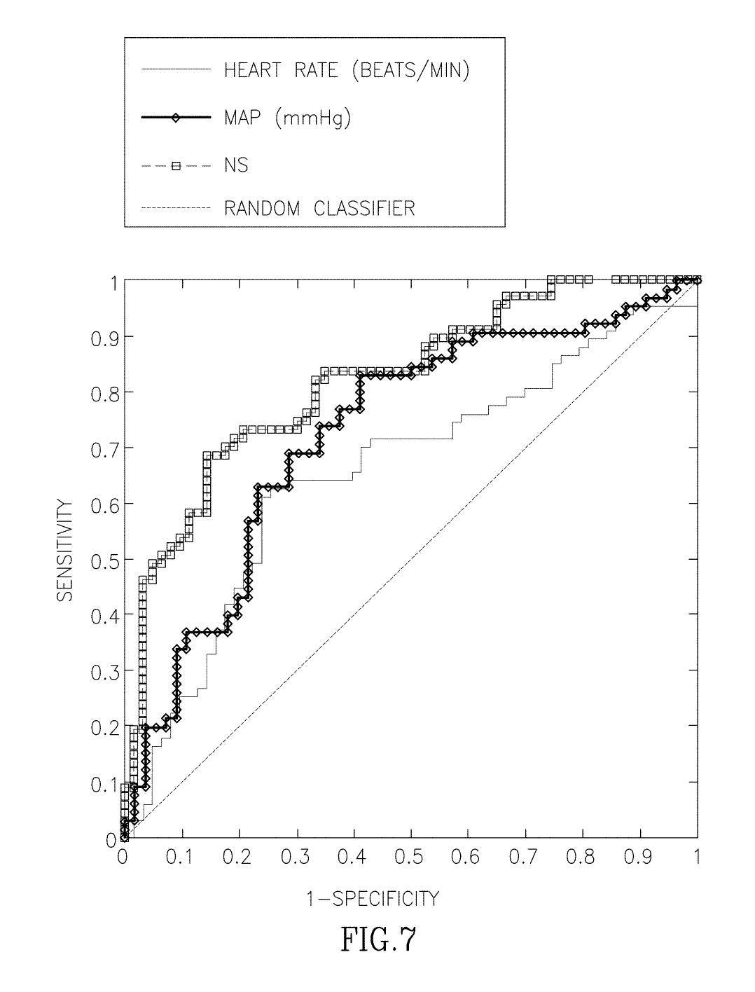

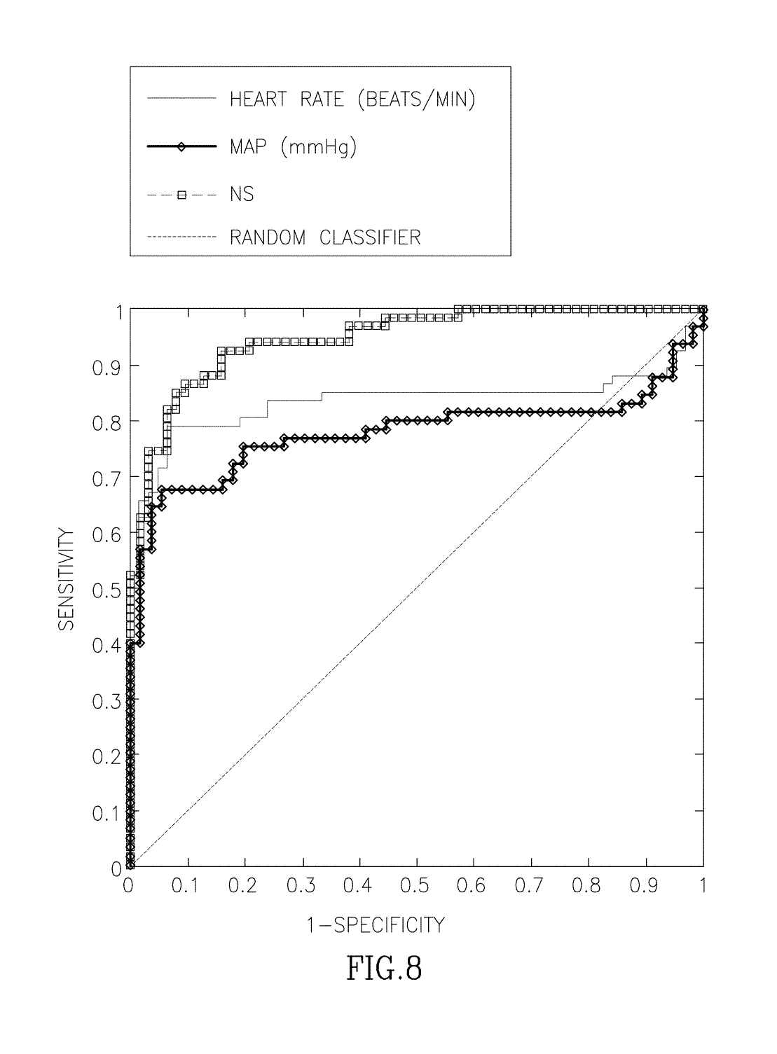

The ROC curves were calculated (n=71) for HR, MAP and NS (all after stimulation) and ΔHR, ΔMAP and ΔNS and are shown in FIG. 7 and FIG. 8. ROC area-under the curve (AUC) sensitivity values at a specificity of 75% are shown in Table 2.

TABLE 2
ROC-AUC, sensitivity, specificity, positive predictive
value (PPV) and negative predictive value (NPV) of the
NS, ΔNS, HR, ΔHR, MAP and ΔMAP at a specificity of 75%.

| Variable | AUC* | Sensitivity | Specificity | PPV | NPV |
|---|---|---|---|---|---|
| HR | 0.66 (0.56-0.75) | 63% | 75% | 72% | 65% |
| MAP | 0.73 (0.64-0.81) | 63% | 75% | 75% | 64% |
| NS | 0.82 (0.75-0.89)^{¶} | 73% | 75% | 75% | 72% |
| ΔHR | 0.84 (0.77-0.91) | 84% | 75% | 78% | 81% |
| ΔMAP | 0.78 (0.70-0.86) | 75% | 75% | 78% | 72% |
| ΔNS | 0.95 (0.91-99)^{#} | 94% | 75% | 80% | 92% |
| Random | 0.50 | 25% | 75% | 50% | 50% |
| classifier | | | | | |

NS, MAP and HR given were obtained after noxious stimulation.
*In parentheses the 95% confidence interval.
^{¶}p = 0.001 vs. HR; p = 0.036 vs. MAP.
^{#}p = 0.0003 vs. ΔHR; p < 0.001 vs. ΔMAP; p = 0.0001 vs. ΔNS; p < 0.0001 vs. HR; p < 0.0001 vs. MAP.

ΔNS outperformed all other variables in its ability to discriminate between noxious (intubation or incision) and non-noxious events with an AUC of 0.95 (95% CI 0.91 to 0.99). The ΔNS AUC was significantly larger compared to all other variables (p=0.0003 vs. ΔHR; p<0.0001 vs. ΔMAP; p<0.0001 vs. HR; p=0.00004 vs. MAP). Moreover, NS after stimulation outperformed MAP and HR in classifying noxious stimuli (AUC 0.82, 95% CI 0.75 to 0.89, p=0.001 vs. HR; p=0.035 vs MAP). The NS outperformed HR and MAP and ΔNS outperformed ΔHR and ΔMAP in terms of sensitivity, specificity and positive and negative predictive values for the detection of noxious stimuli (Table 1). For NS a cut-off value between noxious and non-noxious stimuli of 16 yielded a specificity and sensitivity of 80% and 73%.

These results show that NS can characterize the degree of pain experienced by a subject objectively as well as differentiate between different levels of pain.

### Response to Intubation Under Different Remifentanil Target Concentrations

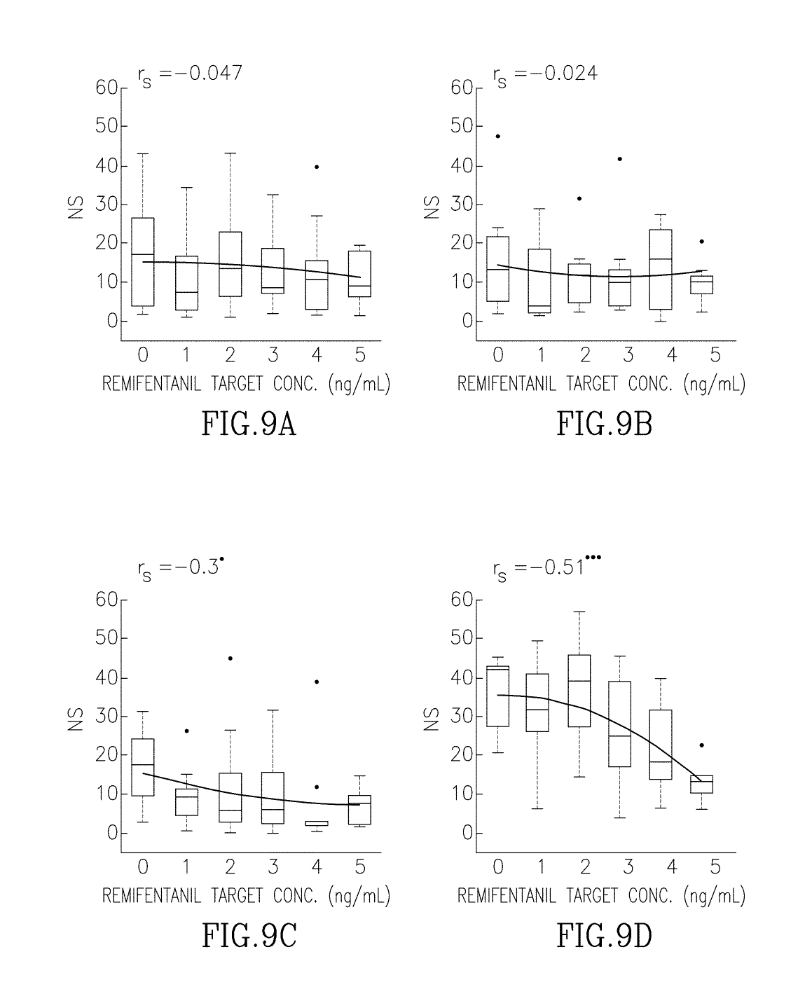

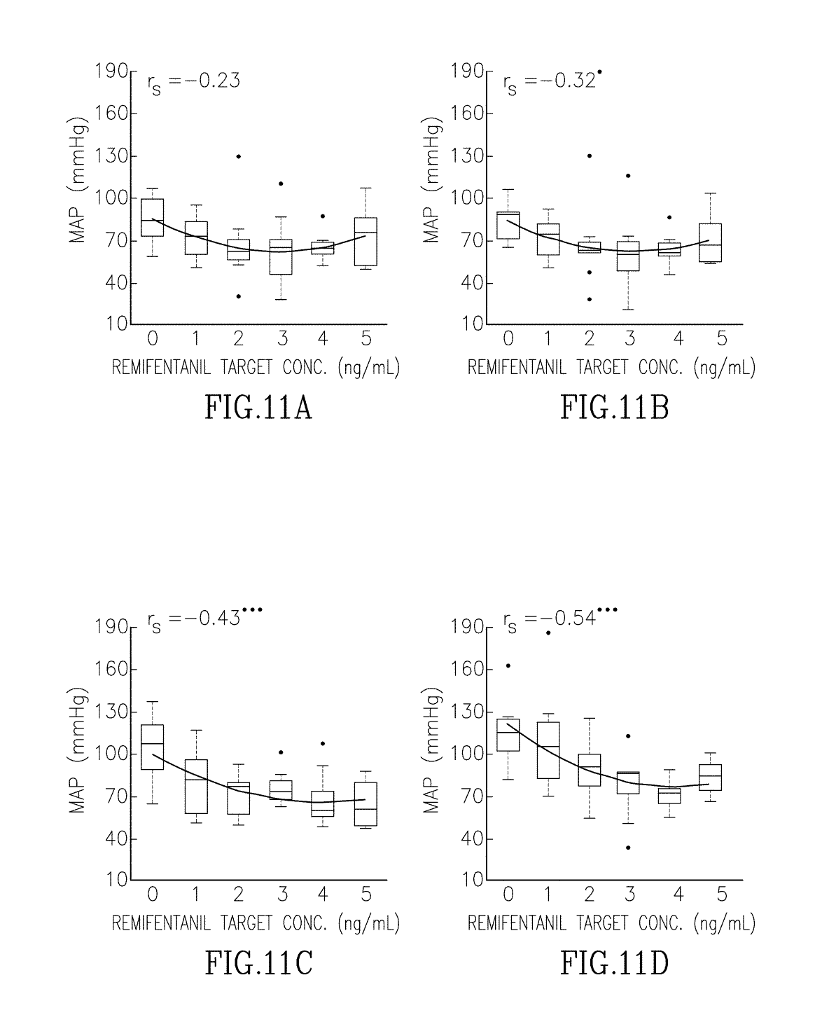

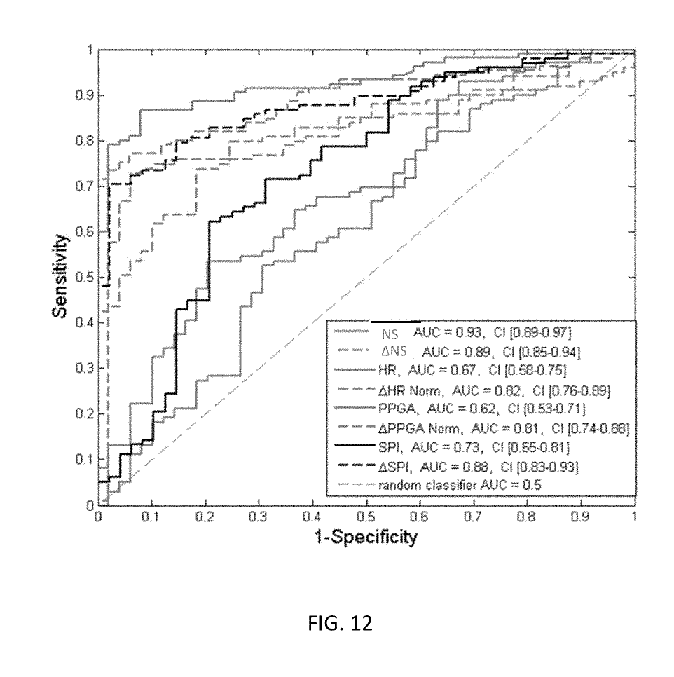

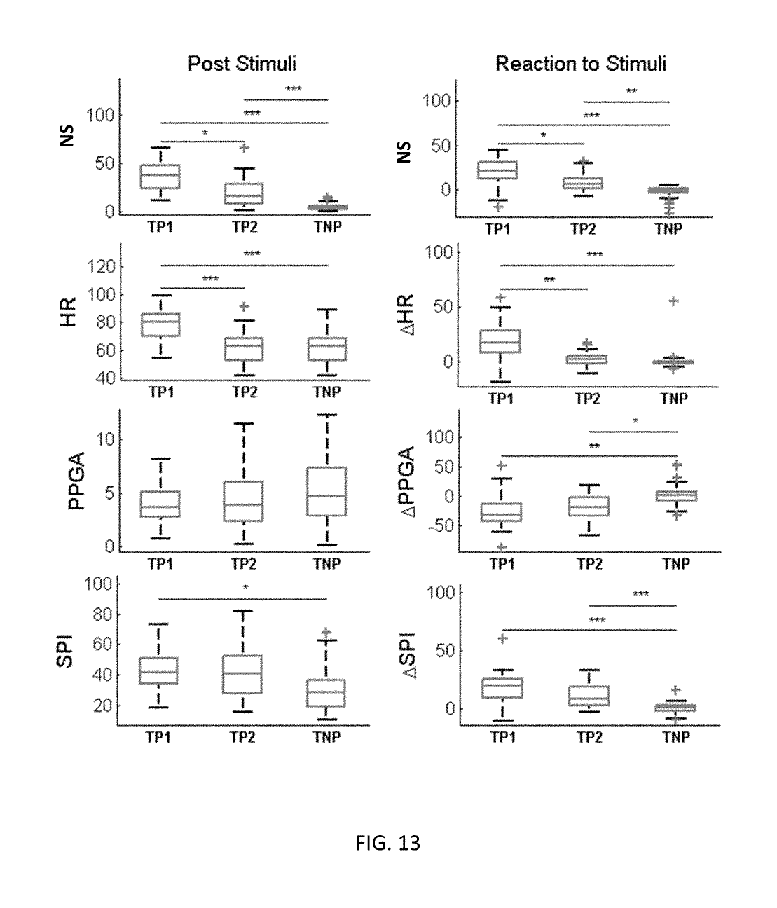

The effects of increasing concentrations of remifentanil on HR (n=57), MAP (n=50) and NS (n=57) before and after noxious stimulation are shown in FIG. 9 to FIG. 11. The NS before and after non-noxious stimulation showed no significant correlation to the remifentanil concentration (rS=−0.047 and 0.024, p>0.05; FIG. 9A and FIG. 9B). The before and after intubation NS values showed a significant Spearman correlation with rS=−0.3, p<0.05 (before, FIG. 9C) and rS=−0.51, p<0.001 (after, FIG. 9D). The Spearman correlation is given (rS), with * indicating p<0.05 and *** p<0.001. The analysis shows that with increasing remifentanil concentrations the NS response to intubation decreases significantly with the smallest response observed at a remifentanil target concentration of 5 ng/mL (FIG. 9D). HR before and after non-noxious stimulation and intubation decreased significantly with increasing remifentanil concentrations in the absence of stimulation (p<0.01; FIG. 10A to FIG. 10D). A similar observation was made for MAP before and after non-noxious stimulation and intubation (p<0.05; FIG. 11A to FIG. 11D). These data show that the NS, as opposed to HR and MAP, was unaffected by administration of drugs in the absence of nociceptive stimuli. Furthermore, these result show that the NS enabled to show a correlation between the dose of the analgesic and nociception.

### Example 2

### Clinical Study #2

### Ethics

This study was executed in accordance with the Declaration of Helsinki, in agreement with the guidelines for conducting a clinical investigation set forth in ISO 14155. Additionally, Israel-specific laws and regulations concerning the conduct of clinical studies with medical devices were followed.

The study was approved by the institutional review board (IRB) (IRB number: #0326-12) and amendments were reviewed and approved by the IRB and was commenced only after the approvals from the IRB were obtained. All requirements imposed by the IRB or regulatory authorities were followed.

Subjects were informed by the investigator of the possible benefits, risks, and outcomes of the use of the device. All subjects who were potential study candidates provided written signed and dated informed consent. The subjects received a signed copy of the informed consent form; the original was filed in the investigational site file.

### Description of the Device and the Nociception Scale (NS)

The monitoring device includes a proprietary console and a finger probe. The console is connected to a PC that runs a user interface. The finger probe contains sensors acquiring the photo-plethysmogram (PPG), galvanic skin response (GSR), skin temperature, and a 3-axis accelerometer. By analyzing the recorded signals, the nociception scale is computed. The recorded signals and parameter trends, including the NS, are displayed on the user interface.

The NS is based on a non-linear combination of nociception-related physiological parameters—heart rate (HR), heart rate variability (HRV, at the 0.15-0.4 Hz band power), photo-plethysmograph wave amplitude (PPGA), skin conductance level (SCL), number of skin conductance fluctuations (NSCF), and their time derivatives. The NS was developed to correlate with a reference clinical score of nociception based on calculated opioid concentration and estimated stimulus strength (i.e., the combined index of stimulus and analgesia or CISA). A composite parameter was derived from Random Forest analysis, a non-linear regression method, in which the physiological signals with their derivatives were used as predictor variables and the CISA was used as the observed variable for the non-linear regression models. The estimated multi-parameter composite derived from the regression analyses was scaled from 0 to 100 to produce the NS.

### Selection of Study Population

Subjects were chosen according to the following criteria: Inclusion criteria: signed written informed consent, age 18-75 years, ASA physical status 1-3, elective surgery under general anesthesia. Exclusion criteria: pregnancy or lactation, history of severe cardiac arrhythmias, presence of neuromuscular disease, abuse of alcohol or illicit drugs, history of mental retardation, dementia, psychiatric disorders, allergy to the anesthetic drugs included in study protocol.

### Study Design and Plan

The first part of the study was applied on all recruited subjects and was designed as within subject comparisons in different set-ups. The first set-up included a comparison of subjects' response to two identical experimental tetanic stimulations; the first with no analgesics and the second following a single bolus of opioids (active treatment self-control). The second set-up included comparison of subjects' response to two noxious clinical stimuli (intubation and first skin incision/trocar insertion) using a non-noxious period as reference. The second part of the study was designed to compare subjects' response to first skin incision/trocar insertion at two levels of Remifentanil. Subjects were randomized into one of two groups after intubation (dose comparison concurrent control; Randomized, double-blind).

### Brief Description of Procedure

Subjects were randomized (equal distribution) to one of two groups of Remifentanil TCI (2 ng/ml or 4 ng/ml) base level. Randomization was performed using Matlab software. Remifentanil infusion was started after the time point of intubation.

In the operating room, subjects were connected to a standard subject monitor (CARESCAPE B650 GE healthcare, Helsinki, Finland (including ECG, NIBP, SPO₂, End Tidal CO₂, Surgical Pleth Index (SPI), and Entropy modules; and to the monitoring device disclosed herein.

Standard anesthesia protocol was used:
a. After 1 mg bolus of midazolam, anesthesia was induced by incremental boluses of 50 mg propofol until subject clinically lost consciousness, and entropy (SE) dropped below 60. Before intubation 2 μg/kg fentanyl and 1 mg/kg rocuronium were administered.
b. After the time point of intubation, for the maintenance of analgesia throughout surgery, subjects were randomized to receive one of two levels of remifentanil TCI (2 ng/ml or 4 ng/ml). Both levels are accepted and used as standard of care.
c. Rescue pain medication during surgery: boluses of 0.05 mg fentanyl upon anesthesiologist's decision.
d. All subjects were administered 100 mg tramal approximately 20 minutes prior to end of surgery.

### Nociceptive Stimulations

1. Experimental noxious stimuli: After anesthesia induction with midazolam and propofol all subjects were subjected to two standardized and similar experimental stimuli (Tetanic stimuli, 60 mA, 100 Hz for 20 seconds) before and 3 minutes after a standardized bolus of 2 μg/kg fentanyl. Tetanic stimuli level was chosen according to standards in the field of anesthesia and pain research.
2. Clinical noxious stimuli: Intubation and skin incision/first trocar insertion.

The list of stimuli and their expected nociceptive levels are detailed in table 3.

TABLE 3
Nociceptive stimuli

| Abbreviation | Level of expected pain | Stimulus |
|---|---|---|
| TP1 | Moderate-severe noxious | Intubation |
| | stimulus | |
| TP2 | Mild-moderate noxious | First incision/trocar |
| | stimulus | insertion |
| TET1 | Mild noxious stimulus | Tetanic without analgesia |
| TET2 | Minor noxious stimulus | Tetanic with analgesia |
| TNP | Non-noxious period | Non-noxious period |

### Data Collection

1. The subject's vital and physiological signals were recorded from the subject monitor by S/5TM Collect (GE healthcare, Helsinki, Finland) to an external PC.
2. The subject was connected with sensors to the monitoring device. The signals and the real time-derived nociception scale (NS) values were recorded to an external PC.
3. During anesthesia and surgery, the following events were noted on the Software event log:
   Noxious events defined by the PI.
   Dose and time of administration of medications including: analgesics, hypnotics, muscle relaxants, and any drug affecting the subject's hemodynamics.
   Clinical signs perceived to correlate to pain or arousal (movement, tearing, sweating, and spontaneous breathing when appropriate).
   Any other relevant data according to the anesthesiologist's judgment.

### Statistical Analysis

The following nociception-related physiological parameters and indices collected in the trial were analyzed:
Nociception Scale (NS)
Physiological parameters: Heart Rate (HR), non-invasive blood pressure (NIBP), photo-plethysmograph amplitude (PPGA) (GE Datex Ohmeda, Helsinki, Finland).
Surgical Pleth Index (SPI) (GE Datex Ohmeda, Helsinki, Finland).

For each subject the response around 4 noxious stimuli and 1 non-noxious stimulus was computed and analyzed.

The noxious/non noxious stimuli for analysis are as follows:
First tetanic stimulation without analgesics (TET 1).
Second tetanic stimulation following 2 μg/kg fentanyl (TET2).
Intubation (TP1).
First incision/trocar insertion (TP2).
Non-noxious period (TNP). TNP was annotated before first skin incision/trocar insertion and after intubation, with relation to a non-noxious marking such as cleaning and covering of the subject. A mandatory requirement was to have at least a 5-minute window around annotation (2.5 minutes before and 2.5 minutes after) with no nociception-related event annotation.

From each of the analyzed parameters three measures were extracted for comparison prior (Pre), after (Post), and during (Reaction) to each stimulus:
Pre stimulus value of parameter: the average value of the parameter was calculated in a window of (−60) to (−30) seconds before stimulus.
Post stimulus value of parameter: the average value of the parameter was calculated in a window of (+10) to (+80) seconds after the stimulus for all stimuli.
Post-long stimulus value of parameter: for comparison between two levels of remifentanil after first skin incision/trocar insertion, the average value of the parameter was calculated in a window of (+10) to (+400) seconds (applied after TP2 only).
Reaction (Δ) of parameter:

Statistical analyses were performed using IBM SPSS Statistics for Windows version 22.0 (IBM Corp., Armonk, N.Y.) and MATLAB R2014a scientific software (Mathworks, Inc., Natick, Mass., US). Descriptive statistics are presented for demographic data whereas continuous data are represented by a median and 25th-75th percentiles. Categorical data are presented by a count and percentage.

P-value of 0.05 is regarded as significant. A Bonferroni correction was applied to maintain a type one error over 8 comparisons, i.e., post and reaction values of the NS, HR, PPGA and SPI. Therefore p-value for significance is 0.05/8=0.00625. Hence throughout the report, for both primary endpoints analysis and for the first of the secondary endpoints, p-values of 0.00625 are regarded significant.

For the second of the secondary endpoints analysis a p-value of 0.01 is regarded significant, after Bonferroni correction, since only 5 nociception-related parameters are compared, i.e. NS, NIBP, HR, PPGA, and SPI (0.05/5=0.01).

### Results

The analysis included 58 subjects: 37 males, 21 females. Mean subjects' age was 46.1±13.1 years, mean subjects' BMI was 29.5±7.5. Surgeries included: open and laparoscopic general and gynecological surgery, ear nose & throat surgery, neurosurgery and plastic surgery.

Response to clinical and experimental noxious stimuli

The response of NS and other parameters to clinical stimuli was investigated based on annotations of TP1, TP2 and TNP, per parameter/index. The median values and IQR (25th and 75th percentiles) of each parameter/index pre, post, and as a reaction to the clinical stimuli are presented in Table 4.

TABLE 4
Pre and post median [25th-75th percentile] values and the reaction to clinical and experimental stimuli, per parameter.

| | NS | | HR | | PPGA* | | SPI | |
|---|---|---|---|---|---|---|---|---|
| | Median [25%-75%] | | Median [25%-75%] | | Median [25%-75%] | | Median [25%-75%] | |
| TP1 pre | 15.3 | [4.2-23.1] | 66.7 | [60.2-74.3] | 5.4 | [4.3-7.7] | 24 | [19.8-27.6] |
| TP1 post | 36.2 | [23.8-47.0] | 79.9 | [70.2-85.6] | 3.7 | [2.9-5.1] | 42.5 | [35.2-52.5] |
| TP1 reaction | 21.2 | [10.8-30.2] | 17.0% | [8.0%-26.0%] | −34.0% | [[(−45.8%)-(−13.5%)] | 20 | [10.3-25.9] |
| TP2 pre | 4.8 | [2.7-17.1] | 61.5 | [51.6-66.4] | 4.9 | [3.1-7.1] | 26.7 | [20.6-39.8] |
| TP2 post | 15.5 | [7.2-29.1] | 63.4 | [53.0-68.1] | 3.8 | [2.3-5.5] | 42.6 | [28.2-54.6] |
| TP2 reaction | 5.2 | [0.6-12.9] | 2.2% | [(−1.4%)-5.0%] | −18.0% | [(−36.1%)-(−2.6%)] | 7.9 | [2.6-16.9] |
| TNP pre | 5.3 | [2-12] | 64 | [52-69] | 5 | [3-7.5] | 28 | [21-39] |
| TNP post | 3.7 | [2.1-7.4] | 63 | [52-68] | 4.7 | [2.9-7.6] | 28 | [19-37] |
| TNP reaction | −0.78 | [(−4.3)-1.2] | −1.2% | [(−2.2%)-0] | −0.25% | [(−7.4%)-6.6%] | 0.16 | [(−2.3)-2.3] |
| TET1 pre | 30.1 | [20.0-46.0] | 77.7 | [68.4-83.7] | 1.7 | [0.9-3.5] | 51.7 | [40.8-68.6] |
| TET1 post | 44 | [31.8-53.8] | 79.1 | [68.9-88.5] | 3.3 | [1.8-4.9] | 41.7 | [33.6-46.9] |
| TET1 reaction | 9.5 | [(−2.5)-19.6] | 0.2% | [(−5.4%)-8.3%] | 78.5% | [24.7%-138.7%] | −14.5 | [(−22.1)-3.3)] |
| TET2 pre | 11.6 | [4.9-28.4] | 66.3 | [58.8-74.6] | 4.8 | [3.5-5.8] | 24.6 | [22.5-38.0] |
| TET2 post | 18.8 | [12.0-30.9] | 68.8 | [59.2-75.1] | 4.8 | [3.5-5.8] | 27.8 | [22.5-38.0] |
| TET2 reaction | 5.8 | [(−2.4)-15.8] | 1.6% | [(−2.6%)-6.4%] | −5% | [(−18.1%)-10.1%] | 2.4 | [(−1.4)-7.0] |

*HR, SPI and NS are expected to rise in response to noxious stimuli while PPGA is expected to decrease in response to noxious stimuli.

The reaction of NS in response to intubation (TP1) and first incision/trocar insertion (TP2) was statistically significant (p<0.0001), increasing by a median reaction value of 21.2 and 5.2, respectively, and was non-significant around non-noxious period (TNP).

The reaction of HR was statistically significant only after TP1, increasing by a median reaction value of 17% (p<0.0001). The reduction of HR from a post median value of 64 BPM to 63 BPM around TNP was statistically significant, but with no clinical significance.

The reaction of PPGA and SPI in response to TP1 and TP2 was statistically significant (p<0.0001) and was non-significant around non-noxious period (TNP).

The values of NS and other parameters in response to experimental tetanic stimuli were also analyzed. The NS reaction to TET1 and TET2 was statistically significant (median reaction values 9.5 and 5.8; p=0.0006 and p=0.0051, respectively). HR did not change with statistical significance following both tetanic stimulations. SPI and PPGA's reaction in response to TET1 were opposite to expected, failing to recognize it as a noxious stimulus. SPI increased with statistical significance in response to TET2 (p=0.0025). PPGA changed non-significantly around TET2.

These result show that NS successfully reacted as predicted, changing with statistical significance after noxious clinical stimuli (TP1, TP2), while not reacting during non-noxious period (TNP). In addition, NS increased significantly in response to experimental tetanic stimuli, TET1 and TET2 (median rise of 9.5 and 5.8 points; p=0.0006, p=0.0051, respectively).

In the analysis testing the ability of parameters and indices to discriminate between noxious (TP1, TP2) and non-noxious stimuli (TNP), a predefined study outcome measure of AUC 0.8 was set. NS reached an AUC of 0.93 [CI 0.89-0.97], outperforming AUC of all other parameters/indices, including SPI, ΔSPI, HR, ΔHR, PPGA and ΔPPGA (with the lower confidence interval for NS (0.89) being higher than the mean AUCs of those parameters). ΔNS reached an AUC of 0.89 [CI 0.85-0.94], also higher than all other tested parameters/indices.

### Discriminate Between Clinical Noxious Stimuli and Non-Noxious Periods

All 58 valid subjects were included in this analysis. FIG. 12 presents the ability of the NS index to discriminate clinical noxious stimuli (TP1, TP2) from non-noxious periods (TNP) using ROC curve analysis. The area under the curve (AUC) was used to compare the performance of NS to the other parameters/index.

The AUC for NS and ΔNS was 0.93 [CI 0.89-0.97]) and 0.89 [CI 0.85-0.94], respectively, higher than the predefined study outcome measure of 0.8.

Other tested parameters reached lower AUCs: HR (0.67 [CI 0.58-0.75]), ΔHR (0.82 [CI 0.76-0.89]), PPGA (0.62 [CI 0.53-0.71]), ΔPPGA (0.81 [CI 0.74-0.88]), SPI (0.73 [CI 0.65-0.81]), and ΔSPI (0.88 [CI 0.83-0.93]).

The sensitivity achieved by NS (86.67% [CI: 78.64%-92.51%]) and ΔNS (80.00% [CI 71.07%-87.17%]) was higher than all other individual parameters/indices tested at specificity of 84%, as listed below in descending order: ΔHR 75.76% [CI 66.42%-83.59%], ΔSPI 79.59% [CI 70.62%-86.83%]), ΔPPGA 63.64% [CI 53.68%-72.80%]), HR 37.37% [CI 28.12%-47.35%]), SPI 42.86% [CI 33.24%-52.89%]) and PPGA 21.21% [CI 35.18%-54.91%]).

### Grading the Response to Different Noxious Stimuli

A repeated measures model was applied for grading the response of the following parameters in the post stimuli window: NS, HR, PPGA and SPI. The model assumed a relation such that TP1 is more intense than TP2 and both are more intense than TNP. For each parameter, only subjects with full sets of annotations for all three stimuli were included in analysis: 44, 42, 42 and 41 subjects for NS, HR, PPGA, and SPI, respectively. A Friedman test was conducted to look for a significant statistical difference between TP1, TP2 and TNP. In case of p-value<0.00625 a post hoc test (Dunn-Sidak) was conducted to verify which of the two groups differ.

Based on the analysis presented in Table 5 below, both NS and ΔNS were the only indices to successfully grade the response to clinical stimuli as expected: TP1>TP2>TNP.

TABLE 5
Grading the response to clinical stimuli TP1, TP2, TNP
by post and reaction (Δ) values. Freidman test analysis.

| | Post | | | | Reaction | | | |
|---|---|---|---|---|---|---|---|---|
| | NS | HR | PPGA | SP1 | ΔNS | ΔHR | ΔPPGA | ASP1 |
| Friedman | <.00001 | <.00001 | .017 | .0012 | <.00001 | <.00001 | <.00007 | <.00001 |
| p-value | | | | | | | | |
| TP1 vs | 0.0013 | <.00001 | .61929 | .92636 | .00415 | .0001 | .92872 | .10385 |
| TP2 | | | | | | | | |
| TP1 vs. | <.00001 | <.00001 | .01361 | .00185 | <.00001 | <.00001 | .00016 | <.00001 |
| TNP | | | | | | | | |
| TP2 vs. | .0001 | .99485 | .22348 | .1222 | .00037 | .03583 | .00144 | .00003 |
| TNP | | | | | | | | |

*p-value < 00625 (after Bonferroni correction .05/8), a Post Hoc (Dunn-Sidak) test was conducted to verify which of the two groups differ.
Significant p-values are in bold.

HR and ΔHR successfully graded TP1>TP2 and TP1>TNP, but failed to grade TP2>TNP. PPGA failed to grade the three events. ΔPPGA significantly graded TP1>TNP and TP2>TNP, but failed to grade TP1>TP2. SPI successfully graded TP1>TNP, but failed to grade TP2>TNP and TP1>TP2. ASPI successfully graded TP1>TNP and TP2>TNP, but failed to grade TP1>TP2.

FIG. 13 shows the relationship between post median [25%-75%] values and reaction median [25%-75%] values for the three clinical stimuli, per parameter (* p<0.00625, ** p<0.001, ***p<0.0001).

Different reaction to a noxious stimulus under different levels of analgesics

To assess the effect of two analgesic doses in response to TP2 (first incision/trocar insertion), subjects were randomized between two groups of remifentanil TCI: 2 ng/ml (low dose) and 4 ng/ml (high dose). There was no difference in demographic and other characteristics between groups. A post-long stimulus window of +10 to +400 seconds was chosen for this analysis aiming to include at least 4 trocar insertions and skin incision with underlying tissue separation. This was the only analysis allowing the inclusion of NIBP in the model due to the relatively long window allowing collection of a sufficient number of data points (sampled every 3-5 minutes). Post-long TP2 values were compared between the two groups of analgesia by Mann-Whitney U test, expecting a lower value in the higher analgesic dose.

The results are presented in table 6 below.

TABLE 6
Reflection of different levels of analgesics during noxious (post-long TP2) period.

| | | | | | Comparison | Pre post |
|---|---|---|---|---|---|---|
| | | | TP2 post- | | 2 vs. 4 Mann | within dose |
| | Remifentanil | TP2 pre | long | TP2 reaction | Whitney test | Wilcoxon |
| | dose | Median | Median | Median | Post-long | sign rank |
| | ng/ml | [25%-75%] | [25%-75%] | [25%-75%] | p-value | test |
| NS | 2 | 10.84 | 21.67 | 4.965 | 0.0069 | 0.0027 |
| (abs) | | [3-17.72] | [12.86-31.24] | [1.17-14.97] | | |
| | 4 | 3.077 | 11.38 | 3.404 | | 0.014 |
| | | [2.171-8.646] | [4.929-18.89] | [(−1.07)-10.11] | | |
| HR | 2 | 62.01 | 64.11 | 3.35% | 0.27 | 0.073 |
| (BPM, | | [54.59-68.87] | [54.75-71.29)] | [(−.72%)-6.49%] | | |
| %) | | | | | | |
| | 4 | 61.21 | 63.16 | 1.16% | | 0.47 |
| | | [51.66-66.36] | [52.2-66.83] | [(−3.07%)-2.39%] | | |
| PPGA | 2 | 4.411 | 4.071 | (−25.34%) | 0.13 | 0.0009 |
| (abs, %) | | [1.791-6.612] | [1.206-5.191] | [(−103.2%)-(−7.38%] | | |
| | 4 | 4.998 | 4.189 | (−3.62%) | | 0.07 |
| | | [3.15-7.628] | [2.828-7.112] | [(−40.89%)-5.15%] | | |
| SPI | 2 | 35.71 | 48.76 | 8.294 | 0.027 | 0.001 |
| (abs) | | [23.34-48-17] | [30.62-62.72] | [2.31-22.51] | | |
| | 4 | 24.91 | 33.14 | 3.562 | | 0.03 |
| | | | [23.04-44.53] | [(−2.09)-13.27] | | |
| NIBP | 2 | 88 | 103.7 | 10% | 0.16 | 0.0003 |
| (mmHg, | | [84.28-96.05] | [92.69-108.8] | [4.2%-15.69% | | |
| %) | | | | | | |
| | 4 | 83 | 92.92 | 3.8% | | 0.01 |
| | | [74-101.8] | 183.13-1021 | [(−.067%)-11.93%] | | |

Statistical significance: p < 01 (after Bonferroni correction).

Only NS reached significance with a post-long stimulus median value of 21.67 (low dose) vs. 11.38 (high dose) (p=0.0069). A paired within-dose comparison of the reaction (ATP2) revealed a significant reaction in the low dose group combined with a non-significant reaction in the high dose group for NS, PPGA, SPI and NIBP. HR did not react significantly to TP2 at any analgesic dose, remaining between 61 and 64 BPM. These results shows the ability of the NS to identify nociception in patients administered with a below optimal dose of an analgesic as well as discriminating between different levels of nociception in correlation with the dose of the analgesic administered.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

Unless specifically stated otherwise, as apparent from the following discussions, it is appreciated that throughout the specification discussions utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A nociception monitoring device comprising:
at least one sensor adapted to sense at least three physiological parameters of a patient, and
a computing unit adapted to:
receive said at least three physiological parameters from said at least one sensor;
compute a nociception scale (NS) value based on an analysis of said at least three physiological parameters and/or features derived therefrom, wherein said NS value is indicative of a nociception level of the patient; and wherein said NS value is essentially unaffected by vasodilating and/or bradycardial effects caused by administration of an analgesic and/or an anesthetic to the patient.

2. The device of claim 1, wherein when said NS value crosses a first predefined threshold value a mild nociception level is identified; and wherein when said NS value crosses a second predefined threshold value a severe nociception level is identified.

3. The device of claim 1, wherein an essentially unaffected NS value is an NS value changed by less than 10 units.

4. The device of claim 1, wherein said patient is anesthetized or administered with an analgesic.

5. (canceled)

6. The device of claim 4, wherein said NS value enables differentiation between no nociception, mild nociception and/or severe nociception in said patient administered with said analgesic.

7. (canceled)

8. The device of claim 4, wherein said NS value provides regression of the nociception level into a numerical scale.

9. The device of claim 1, wherein said at least one sensor is selected from a biopotential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gram (EGG), a laser doppler velocimeter (LDV), a skin temperature sensor, an internal body temperature sensor, a respiration sensor, a capnograph, a pupil diameter monitor, a us blood pressure sensor, a three-axis accelerometer, a diffused correlation spectroscopy (DCS) sensor, an acoustics sensor, a bio-impedance sensor and a piezoelectric sensor, an audio sensor, motion sensing input device or any combination thereof.

10. The device of claim 9, wherein said at least one sensor is selected from a galvanic skin response (GSR) sensor, a plethysmography (PPG) sensor, a skin temperature sensor, and a three-axis accelerometer or any combination thereof

11. The device of claim 10, wherein said at least one sensor comprises at least a galvanic skin response (GSR) sensor and a plethysmography (PPG) sensor.

12. The device of claim 1, comprising at least three sensors; wherein said at least three sensors comprise at least a galvanic skin response (GSR) sensor, a plethysmography (PPG) sensor and a skin temperature sensor, or at least a galvanic skin response (GSR) sensor, a plethysmography (PPG) sensor and a three-axis accelerometer.

13. (canceled)

14. (canceled)

15. The device of claim 1, wherein said at least three parameters are selected from heart rate (HR), heart rate variability (HRV) monitor, amplitude of photoplethysmogram, skin conductance level (SCL), number of skin conductance fluctuations (NSCF), blood pressure, movement and any combination thereof.

16. (canceled)

17. (canceled)

18. (canceled)

19. The device of claim 1, wherein said NS value enables differentiation between no nociception and nociception with a sensitivity of above 80% at a specificity of at least 75%.

20. The device of claim 1, wherein said NS value enables differentiation between no nociception, and nociception with a sensitivity of above 85% at a specificity of at least 75%.

21. (canceled)

22. (canceled)

23. (canceled)

24. The device of claim 1, wherein said NS value enables differentiation between no nociception and mild nociception with a sensitivity of above 75% at a specificity of at least 75%.

25. A device for determining analgesic efficacy comprising:
at least one sensor adapted to sense at least three physiological parameters of a patient before (A0) and after (A1) administration of an analgesics and before (S0) and after (S1) providing a noxious stimuli; and
a computing unit adapted to:
receive said at least three physiological parameters from said at least one sensor obtained for A0, A1, S0, S1 and/or combinations thereof;
compute nociception scale (NS) values for A0, A1, S0, S1 and/or combinations thereof based on an analysis of said at least three physiological parameters and/or features derived therefrom, and determine the efficacy of said analgesic based on a comparison of the nociception scale (NS) values obtained at A0, A1, S0, S1 and/or combinations thereof.

26. The device of claim 25, wherein determining the efficacy of said analgesics comprises determining an efficient dose of said analgesics.

27. The device of claim 25, wherein said NS value is essentially unaffected by vasodilating and/or bradycardial effects of analgesics and/or anesthetics.

28. The device of claim 25, wherein said at least one sensor is selected from biopotential sensor, a galvanic skin response (GSR) sensor, a plethysmography (PPG) sensor, an electrocardiograph (ECG), an electrooculograph (EOG), an electromyograph (EMG), a facial electromyograph (FEMG)/electroencephalograph (EEG), an electro-gastro-gran (EGG), a laser doppler velocimeter (LDV), a skin temperature sensor, an internal body-temperature sensor, a respiration sensor, a capnograph, a pupil diameter monitor, a us blood pressure sensor, a three-axis accelerometer, a diffused correlation spectroscopy (DC S) sensor, an acoustics sensor, a bio-impedance sensor and a piezoelectric sensor, an audio sensor, motion sensing input device or any combination thereof.

29. The device of claim 25, comprising at least three sensors, wherein said at least three sensors comprise a PPG sensor, a GSR sensor and a three-axis accelerometer and/or a skin temperature sensor.

30. (canceled)

31. The device of claim 25, wherein said at least three physiological parameters are selected from heart rate (HR), heart rate variability (HRV) monitor, amplitude of photoplethysmogram, skin conductance level (SCL), number of skin conductance fluctuations (NSCF), arterial blood pressure, movement and any combination thereof.
